(19) United States
(12) Patent Application Publication
(10) Pub. No.: US 2005/0202013 A1
(43) Pub. Date: Sep. 15, 2005

(54) Novel Th2-specific molecules and uses thereof

(75) Inventors: Anthony J. Coyle, Boston, MA (US); Stephen Manning, Arlington, MA (US); Sophie Lehar, Seattle, WA (US); Jose-Carlos Gutierrez-Ramos, Swampscott, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 11/120,777

(22) Filed: May 3, 2005

Related U.S. Application Data
(63) Continuation of application No. 09/989,545, filed on Nov. 20, 2001, now abandoned.
Application No. 09/989,545 is a continuation of application No. 09/413,136, filed on Oct. 6, 1999, now abandoned.
Application No. 09/413,136 is a continuation-in-part of application No. 09/258,670, filed on Feb. 26, 1999, now abandoned.
Application No. 09/258,670 is a continuation-in-part of application No. 09/168,229, filed on Oct. 7, 1998, now abandoned.

(51) Int. Cl. G01N 33/567; C07H 21/04; A61K 39/395; C12N 15/09; C07K 14/74; C07K 16/28

(52) U.S. Cl. 424/144.1; 435/69.1; 435/320.1; 435/372; 530/350; 530/388.22; 536/23.5; 435/7.21

(57) ABSTRACT

Novel Th2-specific polypeptides, proteins, and nucleic acid molecules are disclosed. In addition to isolated, full-length Th2-specific proteins, the invention further provides isolated Th2-specific fusion proteins, antigenic peptides, and anti-Th2-specific antibodies. The invention also provides Th2-specific nucleic acid molecules, recombinant expression vectors containing a nucleic acid molecule of the invention, host cells into which the expression vectors have been introduced, and nonhuman transgenic animals in which a Th2-specific gene has been introduced or disrupted. Diagnostic, screening, and therapeutic methods utilizing compositions of the invention are also provided.

### CROSS-REFERENCE TO RELATED APPLICATION

This application is a Continuation-in-Part of U.S. application Ser. No. 09/258,670, filed Feb. 26, 1999, which is a Continuation-in-Part of U.S. application Ser. No. 09/168,229, filed Oct. 7, 1998, both entitled “Novel Th2-Specific Molecules and Uses Thereof”. The specifications of the copending applications are incorporated herein by reference.

### FIELD OF THE INVENTION

The invention relates to novel Th2-specific nucleic acid sequences and proteins. Also provided are vectors, host cells, and recombinant methods for making and using the novel molecules.

### BACKGROUND OF THE INVENTION

Two distinct types of T lymphocytes are recognized: CD8⁺ cytotoxic T lymphocytes (CTLs) and CD4⁺ helper T lymphocytes (Th cells). CTLs recognize and kill cells that display foreign antigens on their surfaces in conjunction with class I major histocompatibility complex (MHC) molecules. This recognition process triggers the activation, maturation, and proliferation of the precursor CTLs, resulting in CTL clones capable of destroying the cells recognized as foreign.

T cell activation involves a two-step process. An antigen-specific signal is generated by the TCR/CD3 complex, defining the specificity of recognition, followed by a second signal (CD28) delivered by an accessory cell thought to regulate lymphokine expression and proliferation (Meuller et al. (1989) Ann. Rev. Immunol. 7:445; Kohno et al. (1990) Cell. Immunol. 131:1). CD28 is a disulfide-linked homodimer of 44 kDa expressed on the surface of CD4⁺ and CD8⁺ thymocytes (Martin et al. (1986) J. Immunol. 136:3282) and the majority of T cells (Hara et al. (1985) J. Exp. Med. 161:1513). Structurally, CD28 is comprised of a single immunoglobulin-like domain and a 51 amino acid cytoplasmic tail (Aruffo et al. (1987) Proc. Natl. Acad. Sci. USA 84:8573). CD28 signaling initially stabilizes mRNA for various lymphokines, followed by an increase in transcription (Lindsten et al. (1989) Science 244:339).

Th cells are involved in both humoral and cell-mediated forms of effector immune responses. The cell-mediated, or cellular, immune response functions to neutralize microbes that inhabit intracellular locations. Foreign antigens, such as, for example, viral antigens, are synthesized within infected cells and presented on the surfaces of such cells in association with class I MHC molecules leading to the stimulation of the CD8⁺ class I MHC-restricted CTLs. With respect to the humoral, or antibody, immune response, antibodies are produced by B lymphocytes through interactions with Th cells. Specifically, extracellular antigens are endocytosed by antigen-presenting cells (APCs), processed, and presented preferentially in association with class II MHC molecules to CD4⁺ class II MHC-restricted Th cells. These Th cells in turn activate B lymphocytes, resulting in antibody production.

During the course of an immune response, T cells differentiate into Th phenotypes defined by their pattern of cytokine secretion and immunomodulatory properties (Abbas et al. (1996) Nature 383:787). Th cells are composed of at least two distinct subpopulations, termed Th1 and Th2 cell subpopulations (Mosmann et al. (1989) Ann. Rev. Immunol. 7:145; Del Prete et al. (1991) J. Clin. Invest. 88:346; Wiernenga et al. (1990) J. Immunol. 144:4651; Yamamura et al. (1991) Science 254:277; Robinson et al. (1993) J. Allergy Clin. Immunol. 92:313). Th1 and Th2 cells appear to function as part of the different effector functions of the immune system (Mosmann et al. (1989) Ann. Rev. Immunol. 7:145). Specifically, Th1 cells direct the development of cell-mediated immunity, triggering phagocyte-mediated host defenses, and are associated with delayed hypersensitivity. Accordingly, infections with intracellular microbes tend to induce Th1-type responses. Th2 cells drive humoral immune responses, which are associated with, for example, defenses against certain helminthic parasites, and are involved in antibody and allergic responses.

Th1 cells secrete interleukin-2 (IL-2), interferon-γ (IFN-γ), and tumor neucrosis factor-α (TNF-α). These cytokines enhance inflammatory cell-mediated responses and have a pathogenic role in the development of autoimmune disease. Th2 cells secrete interleukin-4 (IL-4), interleukin-5 (IL-5), interleukin-10 (IL-10), and interleukin-13 (IL-13). These cytokines suppress inflammatory responses while potentiating humoral immunity and control and reverse disease evolution (Scott et al. (1994) Immunity 1:73; Smith et al. (1998) J. Immunol. 160:4841; Abbas et al. (1996) Nature 383:787). The different type of cytokines released upon stimulation has been demonstrated to be central to disease evolution (Chu and Londei (1996) J. Immunol. 157:2685; Hsieh et al. (1993) Science 260:547).

In vivo treatment with soluble CD28 antagonists in animal models can suppress transplant rejection and autoimmunity. This inhibitory effect was first demonstrated to be the result of inhibition of IL-2 production and clonal expansion required to generate inflammatory responses (Padrid et al. (1998) Am. J. Respir. Cell. Mol. Biol. 18:453; and Lenschow et al. (1992) Science 257:789). Recently, studies have indicated that differential signaling through the CD28 receptor has distinct effects on the production of Th2 cytokines, including II-4 and IL-5. Generally, CD28 signaling can enhance the development of Th2 cells by regulating IL-4 production but is not essential for the development of Th1 cells (Reilifson et al. (1997) J. Immunol. 158:658).

Once Th1 and Th2 subpopulations are expanded, the cell types tend to negatively regulate one another through the actions of cytokines unique to each. For example, Th1-produced IFN-γ negatively regulates Th2 cells, while Th2-produced IL-10 negatively regulates Th1 cells. Moreover, cytokines produced by Th1 and Th2 antagonize the effector functions of one another (Mosmann et al. (1991) Immunol. Today 12:49). Furthermore, the induction and maintenance of tolerance in both transplant and autoimmune diseases is a direct consequence of enhanced Th2 activity at the expense of Th1 cells (Strom et al. (1996) Curr. Opin. Immunol. 8:688).

Failure to control or resolve an infectious process often results from an inappropriate, rather than an insufficient immune response, and can underlie a variety of distinct immunological disorders. Such disorders can include, for example, atopic conditions (i.e., IgE-mediated allergic conditions) such as asthma, allergy, including allergic rhinitis, dermatitis, including psoriasis, pathogen susceptibilities, chronic inflammatory disease, organ-specific autoimmunity, graft rejection, and graft versus host disease. For example, nonhealing forms of human and murine leishmaniasis result from strong but counterproductive Th2-like-dominated immune responses. Lepromatous leprosy also appears to feature a prevalent, but inappropriate, Th2-like response. A drop in the ratio of Th1-like cells to other Th cell subpopulations may play a critical role in the progression toward disease symptoms in HIV infection.

Further, while Th1-mediated inflammatory responses to many pathogenic microorganisms are beneficial, such responses to self antigens are usually deleterious. It has been suggested that the preferential activation of Th1-like responses is central to the pathogenesis of such human inflammatory autoimmune diseases as multiple sclerosis and insulin-dependent diabetes. For example, Th1-type cytokines predominate in the cerebrospinal fluid of patients with multiple sclerosis, pancreases of insulin-dependent diabetes patients, thyroid glands of Hashimoto's thyroiditis, and gut of Crohn's disease patients, suggesting that such patients mount a Th1-like, not a Th2 -like, response to the antigen(s) involved in the etiopathogenesis of such disorders.

The profile of the natural immune response, specifically cytokine production by natural killer cells or cells of basophil lineage, may determine the phenotype of the subsequent immune response. Therefore, methods are needed to regulate an immune response, particularly to modulate a Th1 or Th2 response. Genes and proteins differentially expressed between the two subsets of T cells may play a role in determining the phenotype of the subsequent immune response.

### SUMMARY OF THE INVENTION

Isolated nucleic acid molecules corresponding to Th2-specific nucleic acid sequences are provided. Additionally amino acid sequences corresponding to the polynucleotides are encompassed. In particular, the present invention provides for isolated nucleic acid molecules comprising nucleotide sequences encoding the amino acid sequences shown in SEQ ID NOs:2, 4, 6, 8, 10, 12, 14, and 16 or the nucleotide sequences encoding the DNA sequence deposited in a bacterial host as ATCC Accession Number 203302, ATCC Accession Number 203569, and ATCC Accession Number 203797. Further provided are Th2-specific polypeptides having an amino acid sequence encoded by a nucleic acid molecule described herein.

The present invention also provides vectors and host cells for recombinant expression of the nucleic acid molecules described herein, as well as methods of making such vectors and host cells and for using them for production of the polypeptides or peptides of the invention by recombinant techniques.

The Th2-specific molecules of the present invention are useful for modulating the phenotype of immune and respiratory responses, particularly for regulating a Th1 or Th2 response. The molecules are useful for the diagnosis and treatment of immune and respiratory disorders, particularly for the treatment and diagnosis of T-lymphocyte-related disorders, including, but not limited to, atopic conditions, such as asthma and allergy, including allergic rhinitis, psoriasis, the effects of pathogen infection, chronic inflammatory diseases, organ-specific autoimmunity, graft rejection, and graft versus host disease. Accordingly, in one aspect, this invention provides isolated nucleic acid molecules encoding Th2-specific proteins or biologically active portions thereof, as well as nucleic acid fragments suitable as primers or hybridization probes for the detection of Th2-specific-encoding nucleic acids.

Another aspect of this invention features isolated or recombinant Th2-specific proteins and polypeptides. Preferred Th2-specific proteins and polypeptides possess at least one biological activity possessed by naturally occurring Th2-specific proteins.

Variant nucleic acid molecules and polypeptides substantially homologous to the nucleotide and amino acid sequences set forth in the sequence listings are encompassed by the present invention. Additionally, fragments and substantially homologous fragments of the nucleotide and amino acid sequences are provided.

Antibodies and antibody fragments that selectively bind the Th2-specific polypeptides and fragments are provided. Such antibodies are useful in detecting the Th2-specific polypeptides as well as in regulating the T-cell immune response.

In another aspect, the present invention provides a method for detecting the presence of Th2-specific activity or expression in a biological sample by contacting the biological sample with an agent capable of detecting an indicator of Th2-specific activity such that the presence of Th2-specific activity is detected in the biological sample.

In yet another aspect, the invention provides a method for modulating Th2-specific activity comprising contacting a cell with an agent that modulates (inhibits or stimulates) Th2-specific activity or expression such that Th2-specific activity or expression in the cell is modulated. In one embodiment, the agent is an antibody that specifically binds to Th2-specific protein. In another embodiment, the agent modulates expression of Th2-specific protein by modulating transcription of a Th2-specific gene, splicing of a Th2-specific mRNA, or translation of a Th2-specific mRNA. In yet another embodiment, the agent is a nucleic acid molecule having a nucleotide sequence that is antisense to the coding strand of the Th2-specific mRNA or the Th2-specific gene.

In one embodiment, the methods of the present invention are used to treat a subject having a disorder characterized by aberrant Th2-specific protein activity or nucleic acid expression by administering an agent that is a Th2-specific modulator to the subject. In one embodiment, the Th2-specific modulator is a Th2-specific protein. In another embodiment, the Th2-specific modulator is a Th2-specific nucleic acid molecule. In other embodiments, the Th2-specific modulator is a peptide, peptidomimetic, or other small molecule.

The present invention also provides a diagnostic assay for identifying the presence or absence of a genetic lesion or mutation characterized by at least one of the following: (1) aberrant modification or mutation of a gene encoding a Th2-specific protein; (2) misregulation of a gene encoding a Th2-specific protein; and (3) aberrant post-translational modification of a Th2-specific protein, wherein a wild-type form of the gene encodes a protein with a Th2-specific activity.

In another aspect, the invention provides a method for identifying a compound that binds to or modulates the activity of a Th2-specific protein. In general, such methods entail measuring a biological activity of a Th2-specific protein in the presence and absence of a test compound and identifying those compounds that alter the activity of the Th2-specific protein.

The invention also features methods for identifying a compound that modulates the expression of Th2-specific genes by measuring the expression of the Th2-specific sequences in the presence and absence of the compound.

Other features and advantages of the invention will be apparent from the following detailed description and claims.

### BRIEF DESCRIPTION OF THE DRAWINGS

FIG. 1 shows the amino acid sequence alignment for the protein (m1022; SEQ ID NO:6) encoded by murine 1022 (SEQ ID NO: 5) and the protein (h1022; SEQ ID NO: 14) encoded by human 1022 (SEQ ID NO: 13) with the Trypanosoma brucei alpha core subunit of DNA polymerase (GenBank Accession Number CAA43286, SEQ ID NO: 17).

FIG. 2 shows the amino acid sequence alignment for the protein (m1228, referred to as mICOS; SEQ ID NO: 10) encoded by murine 1228 (SEQ ID NO: 9) and the protein (h1228, referred to as hICOS; SEQ ID NO: 12) encoded by human 1228 (SEQ ID NO: 11) with the murine glycoprotein CD28 precursor (mCD28 ; GenBank Accession Number AAA37395; SEQ ID NO: 18), the human T-cell-specific surface glycoprotein CD28 precursor (hCD28; SP Accession Number P10747; SEQ ID NO: 19), the murine cytotoxic t-lymphocyte protein 4 precursor (mCTLA-4; SP Accession Number P09793; SEQ ID NO: 20), and the human cytotoxic t-lymphocyte protein 4 precursor (hCTLA-4; GI Accession Number 4885167; SEQ ID NO: 21). The putative transmembrane domain of these sequences is indicated by the box.

FIG. 3 shows the amino acid sequence alignment for the protein (m1419; SEQ ID NO:2) encoded by murine 1419 (SEQ ID NO: 1) and the protein (h1419; SEQ ID NO: 16) encoded by human 1419 (SEQ ID NO: 15) with the Caenorhabditis elegans C54H2.1 gene product (sequence U58728 no. 1326268; GenBank Accession Number AAB00590; SEQ ID NO: 22).

FIG. 4 shows real time PCR analysis (Taqman™) of mICOS expression on resting and activated leukocytes.

FIG. 5 demonstrates that mICOS-Ig (100 μg/ml) (heavy solid line) fails to bind to either mB7-1 or mB7-2 EL-4 transfectants, whereas CTLA-4-Ig (1 μg/ml) (light line) and CD28-Ig (10 μg/ml) (dashed lines) bind at 10- to 100-fold lower concentrations. Cells treated with human Ig (dotted lines) are shown for comparison.

FIG. 6 demonstrates that mICOS-Ig (10 μg/ml) binds to CD40-activated, but not resting B cells (A), and to resting and CD40-stimulated bone marrow-derived dendritic cells (B).

FIG. 7 demonstrates that mICOS signaling is critical for activation of Th2, but not Th1 effector cells. CD4+ T cells from DO 11.10 αβ-TCR transgenic mice were differentiated to Th1 or Th2 effector populations. Cells were then reactivated in the presence of mICOS-Ig (1-100 μg/ml) (open squares) or hIg (closed barsData are shown as the mean±s.e.m. of triplicate wells and is representative of 4 different experiments.

FIG. 8 demonstrates inhibition of humoral responses by ICOS-Ig. Mice were immunized in the footpad with OVA/alum on day 0 boosted s.c. on day 8. Ten days later, blood was taken from the tail vein and sera titers of antigen-specific IgE (A) and IgG1 (B) measured by specific ELISA. Mice were treated i.p. with ICOS-Ig (100 μg/mouse) (closed squares) or hIg as the appropriate control (open squares) on day 7, 8, and 9. Data are shown as the mean±s.e.m. absorbance (O.D.) at serial sera dilution and represent data from n=5-6 individual animals.

FIG. 9 demonstrates inhibition of eosinophilic inflammation of the airways in an active immunization model by ICOS-Ig and CTLA-4Ig. One hour prior to allergen challenge, immunized mice were treated intranasally with 100 μg of either ICOS-Ig (open columns), CTLA-4-Ig (shaded columns) or hIg (closed columns) as the appropriate isotype control. Data are shown as the mean±s.e.m. eosinophils/ml×10³ for n=5 animals. Statistical significance (p<0.05) was determined by a Student's T-test and indicated by *.

FIG. 10 demonstrates attenuation of Th2, but not Th1 mediated mucosal inflammation by mICOS-Ig. Aeroallergen challenge of Th1 (A) or Th2 (B) recipient mice after adoptive transfer results in a neutrophilic or eosinophilic lung inflammatory response, respectively, associated with IFN-γ or IL-5 secretion. Pretreatment with mICOS-Ig or CTLA-4-Ig (open bars) inhibited Th2-mediated inflammation. In contrast, CTLA-4-Ig, but not ICOS-Ig, inhibited Th1 medicated inflammation as compared to mice treated with hIg (closed bars). Data are shown as the mean±s.e.m. of n-4-6 mice and is representative of 3 different experiments. Significance (*) was determined by a Student's T-test and a value of p<0.05 considered significant.

FIG. 11 demonstrates selective suppression of Th2-mediated altered airway hyperresponsiveness by ICOS-Ig. Exposure to OVA in both (A) Th1 and (B) Th2 recipient mice results in increased airway hyperresponsiveness to methacholine (20 mg/ml) as compared to PBS-exposed mice. The baseline response is indicated in the open bars and the maximal response to inhaled methacholine in closed bars. Significance (#) was determined by a Student's T-test and a value of p<0.05 considered significant. Control mice received hIg as the appropriate isotype control. Data are shown as the mean±s.e.m. of n=3-6 mice and is representative of 2 different experiments. Significance from Ig treated mice (*) was determined by a Student's T-test and a value of p<0.05 considered significant.

### DETAILED DESCRIPTION OF THE INVENTION

The present invention provides isolated nucleic acid molecules comprising a nucleotide sequence encoding the Th2-specific polypeptides whose amino acid sequence is given in SEQ ID NO:2, 4, 6, 8, 10, 12, 14, and 16, respectively, or a variant or fragment of the polypeptide. Nucleotide sequences encoding the Th2-specific proteins of the invention are set forth in SEQ ID NOs: 1, 3, 5, 7, 9, 11, 13, and 15.

The present invention relates to methods and compositions for the modulation, diagnosis, and treatment of immune and respiratory disorders, especially T helper (Th) cell and Th cell-like related disorders. Such immune disorders include, but are not limited to, chronic inflammatory diseases and disorders, such as Crohn's disease, reactive arthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease, sarcoidosis, atopic conditions, such as asthma and allergy, including allergic rhinitis, gastrointestinal allergies, including food allergies, eosinophilia, allergen-provoked airway inflammation, and conjunctivitis, glomerular nephritis, certain pathogen susceptibilities such as helminthic (e.g., leishmaniasis), certain viral infections, including HIV, and bacterial infections, including tuberculosis and lepromatous leprosy.

Respiratory disorders include, but are not limited to, apnea, asthma, particularly bronchial asthma and associated airway hyperresponsiveness, reberillium disease, bronchiectasis, bronchitis, bronchopneumonia, cystic fibrosis, diphtheria, dyspnea, emphysema, chronic obstructive pulmonary disease, allergic bronchopulmonary aspergillosis, pneumonia, acute pulmonary edema, pertussis, pharyngitis, atelectasis, Wegener's granulomatosis, Legionnaires disease, pleurisy, rheumatic fever, and sinusitis.

Three novel human genes (h1419, h1022, and h1228), and their murine orthologues (m1419, m1022, and m1228), are provided. The genes are differentially expressed in the Th2 subset of T-helper cells. Such sequences are referred to as “Th2-specific” indicating that the genes are expressed in the Th2 subset of T-helper cells but not expressed, or only expressed at very low levels, in the Th1 subset. The genes are overexpressed in CD3/TCR-activated Th2 cells.

The molecules of the invention (e.g., nucleic acid molecules, polypeptides, antisense molecules, antibodies) can be used to modulate an immune response. By “modulating” is intended the upregulating or downregulating of an immune response. In one embodiment, the immune response is a T cell, e.g., T helper cell, e.g., Th1 and/or Th2 cell, response. T cell, e.g., T helper cell, responses are manifested by, for example, lymphokine production, cellular proliferation, signaling events, and other effector functions. For example, a Th1 cell response can include the production of IL-2, tumor necrosis factor beta, and interferon gamma. Generally, Th1 cells have a pathogenic role in the development of autoimmune disease. In another example, a Th2 cell response can include the production of IL-3, GM-CSF, IL-4, IL-5, IL-10, and/or IL-13 and/or the generation of antibodies, e.g., IgE antibodies (e.g., by modulating the development and/or function of B cells), and/or the production, migration, function and/or differentiation, e.g., terminal differentiation, of eosinophils. Additional Th1 and Th2 cell responses can be found in Anderson and Coyle (1994) Trends in Pharmacological Sciences 15:324-332, the contents of which are hereby incorporated by reference. Typically, Th2 cells control and reverse disease evolution. Thus, modulation of an immune response can involve the modulation of one or more of the Th1 and/or Th2 cell responses described herein.

The first of these Th2-specific genes, h1419, encodes a 384 amino acid protein (SEQ ID NO: 16). The nucleotide sequence for h1419 is provided in SEQ ID NO: 15. Prosite program analysis was used to predict various sites within the h1419 protein. N-glycosylation sites were predicted at amino acids (aa) 214-217, 229-232, and aa 260-263. cAMP- and cGMP-dependent protein kinase phosphorylation sites were predicted at aa 61-64, 258-261, and 308-311. Protein kinase C phosphorylation sites were predicted at aa 47-49, 86-88, 116-118, 158-160, 174-176, 256-258, 281-283, 301-303, and 304-306. Casein kinase II phosphorylation sites were predicted at aa 17-20, 47-50, 130-133, 216-219, 251-254, 281-284, 317-320, 322-325, and 343-346. N-myristoylation sites were predicted at aa 29-34, 112-117, 166-171, 177-182, 183-188, 270-275, and 364-369.

The murine orthologue m1419 encodes a 3.6 Kb transcript having the corresponding cDNA set forth in SEQ ID NO: 1. This transcript encodes a 392 amino acid protein (SEQ ID NO: 2) having a molecular weight of approximately 42.8 kDa (excluding post-translational modifications). N-glycosylation sites were predicted at amino acids (aa) 230-233 and aa 261-264. cAMP- and cGMP-dependent protein kinase phosphorylation sites were predicted at aa 61-64, 259-262, and 316-319. Protein kinase C phosphorylation sites were predicted at aa 47-49, 86-88, 116-118, 158-160, 174-176, 257-259, 289-291, 309-311, and 312-314. Casein kinase II phosphorylation sites were predicted at aa 17-20, 47-50, 130-133, 217-220, 252-255, 325-328, 330-333, and 351-354. N-myristoylation sites were predicted at aa 29-34, 112-117, 166-171, 177-182, 183-188, 271-276, and 372-377. This protein displays homology (at least 40% to about 44% identity over a 140 amino acid overlap) to the predicted protein sequence encoded by a putative Caenorhabditis elegans gene (GenBank Accession Number AAB00590) whose function is unknown.

A plasmid containing the h1419 cDNA insert, designated P1419, was deposited with American Type Culture Collection (ATCC), 10801 University Blvd., Manassas, Va., on Feb. 25, 1999 and assigned Accession Number 203797. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. § 112.

The second of these Th2-specific genes, h1022, encodes a 481 amino acid protein (SEQ ID NO: 14). The nucleotide sequence for h1022 is set forth in SEQ ID NO: 13. An analysis of h1022 predicts that the N-terminal 70 amino acids represent a signal peptide. A transmembrane segment from amino acids (aa) 7-23 was predicted by MEMSAT. Prosite program analysis was used to predict various sites within the h1022 protein. N-glycosylation sites were predicted at aa 293-296 and aa 397-400. N-glycosylation sites were predicted at aa 293-296 and 397-400. Glycosaminoglycan attachment sites were predicted at aa 20-23 and 236-239. Protein kinase C phosphorylation sites were predicted at aa 117-119, 148-150, 176-178, 226-228, 276-278, 430-432, and 466-468. Casein kinase II phosphorylation sites were predicted at aa 143-146, 156-159, 240-243, 248-251, 284-287, 306-309, 449-452, and 466-469. A tyrosine kinase phosphorylation site was predicted at aa 67-73. N-myristoylation sites were predicted at aa 10-15, 112-117, 237-242, 266-271, 406-411, and 420-425. An amidation site was predicted at aa 94-97. An EF-hand calcium-binding domain was predicted at aa 158-171.

The murine orthologue m1022 encodes two transcripts, a shorter 1.6 Kb form having the corresponding murine cDNA set forth in SEQ ID NO: 3, and a longer 4.4 Kb form having the corresponding murine cDNA set forth in SEQ ID NO: 5. Both transcripts encode a 464 amino acid protein (SEQ ID NOs: 4 and 6) having a molecular weight of approximately 54.2 kDa (excluding post-translational modifications. The m1022 protein is homologous (about 20-25% identity overall) to the alpha core subunit of DNA polymerase from Trypanosoma brucei (GenBank Accession Number CAA43286). An analysis of m1022 predicted that the N-terminal 70 amino acids (aa) represent a signal peptide. A transmembrane segment from aa 7-23 was predicted by MEMSAT. Prosite program analysis was used to predict various sites within the m1022 protein. N-glycosylation sites were predicted at aa 276-279 and aa 380-383. A glycosaminoglycan attachment site was predicted at aa 219-222. Protein kinase C phosphorylation sites were predicted at aa 117-119, 147-149, 219-221, 259-261, 413-415, and 449-451. Casein kinase II phosphorylation sites were predicted at aa 142-145, 155-158, 163-166, 267-270, 289-292, 432-435, and 449-452. Tyrosine kinase phosphorylation sites were predicted at aa 67-73 and 223-230. N-myristoylation sites were predicted at aa 6-11, 112-117, 249-254, 389-394, and 403-408. An amidation site was predicted at aa 94-97.

A plasmid containing the h1022 cDNA insert, designated P1022, was deposited with American Type Culture Collection (ATCC), 10801 University Blvd., Manassas, Va., on Jan. 8, 1999 and assigned Accession Number 203569. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. § 112.

The third of these Th2-specific genes, h1228, encodes a 198 amino acid protein (SEQ ID NO: 12). The nucleotide sequence for h1228 is set forth in SEQ ID NO: 11. This protein is an Ig superfamily member with 33% homology to hCD28 and 26% homology to hCLTA-4. Examination of the amino acid sequence revealed 4 conserved cysteine residues (aa 41, 62, 82, and 135 of SEQ ID NO: 12) and a conserved PPP motif ( present as FDPPPF, aa 113-118 of SEQ ID NO: 12) in the extracellular domain, which is common to CD28 and its related homologue CTLA-4. The h1228 sequence additionally contains a YMFM motif (amino acid residues 178-181 of SEQ ID NO: 12) similar to the phosphotyrosine-based motif pYMNM common in CD28 and CTLA-4, which appears to be required for CD28 -mediated PI-3K activity. Signaling through 1228 is required for Th2 cytokine production. Further, inhibition of 1228 in a murine model of asthma attenuates Th2 mucosal inflammation and airway hyperresponsiveness. 1228 effectively replaces the CD28 signal and can provide a costimulatory signal specific for cytokine production from Th2 effector cells.

The murine orthologue m1228 encodes two transcripts, a shorter 2.1 Kb form having the corresponding cDNA sequence set forth in SEQ ID NO: 7, and a longer 3.3 Kb form having the corresponding cDNA sequence set forth in SEQ ID NO: 9. Both transcripts encode a 200 amino acid protein (SEQ ID NOs: 8 and 10) having a molecular weight of approximately 22.7 kDa (excluding post-translational modifications). This protein shares 69% identity with h1228, 36.5% identity with mCD28, and 38.5% identity with mCTLA-4.

A plasmid containing the h1228 cDNA insert, designated P1228, was deposited with American Type Culture Collection (ATCC), 10801 University Blvd., Manassas, Va., on Oct. 2, 1998, and assigned Accession Number 203302. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. § 112.

The Th2-specific sequences of the invention are members of a family of molecules (the “Th2-specific family”) having conserved functional features. The term “family” when referring to the proteins and nucleic acid molecules of the invention is intended to mean two or more proteins or nucleic acid molecules having sufficient amino acid or nucleotide sequence identity as defined herein. Such family members can be naturally occurring and can be from either the same or different species. For example, a family can contain a first protein of murine origin and a homologue of that protein of human origin, as well as a second, distinct protein of human origin and a murine homologue of that protein. Members of a family may also have common functional characteristics.

Preferred Th2-specific polypeptides of the present invention have an amino acid sequence sufficiently identical to the amino acid sequence of SEQ ID NO: 2, 4, 6, 8, 10, 12, 14, or 16. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 95%, or 98% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, only exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to Th2-specific nucleic acid molecules of the invention. BLAST protein searches can be performed with the XBLAST program, score=50, wordlength=3, to obtain amino acid sequences homologous to Th2-specific protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

Accordingly, another embodiment of the invention features isolated Th2-specific proteins and polypeptides having a Th2-specific protein activity. As used interchangeably herein, a “Th2-specific protein activity”, “biological activity of a Th2-specific protein”, or “functional activity of a Th2-specific protein” refers to an activity exerted by a Th2-specific protein, polypeptide, or nucleic acid molecule on a Th2-specific responsive cell as determined in vivo, or in vitro, according to standard assay techniques. A Th2-specific activity can be a direct activity, such as an association with or an enzymatic activity on a second protein, or an indirect activity, such as a cellular signaling activity mediated by interaction of the Th2-specific protein with a second protein. In a preferred embodiment, a Th2-specific activity includes at least one or more of the following activities: (1) modulating (stimulating and/or enhancing or inhibiting) cellular proliferation, differentiation, and/or function, particularly immune cells, for example leukocytes; (2) modulating a Th2-specific immune response; (3) inhibiting a Th1 immune response; (4) inducing and/or maintaining tolerance in both transplant and autoimmune diseases; (5) binding a Th2-specific ligand; or (6) modulating Th2-specific cytokines such as IL-4, Ill-5, IL-10, and IL-13.

An “isolated” or “purified” Th2-specific nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the invention, “isolated” when used to refer to nucleic acid molecules, excludes isolated chromosomes. For example, in various embodiments, the isolated Th2-specific nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A Th2-specific protein that is substantially free of cellular material includes preparations of Th2-specific protein having less than about 30%, 20%, 10%, or 5% (by dry weight) of non-Th2-specific protein (also referred to herein as a “contaminating protein”). When the Th2-specific protein or biologically active portion thereof is recombinantly produced, preferably, culture medium represents less than about 30%, 20%, 10%, or 5% of the volume of the protein preparation. When Th2-specific protein is produced by chemical synthesis, preferably the protein preparations have less than about 30%, 20%, 10%, or 5% (by dry weight) of chemical precursors or non-Th2-specific chemicals.

Various aspects of the invention are described in further detail in the following subsections.

I. Isolated Nucleic Acid Molecules

One aspect of the invention pertains to isolated nucleic acid molecules comprising nucleotide sequences encoding Th2-specific proteins or biologically active portions thereof, as well as nucleic acid molecules sufficient for use as hybridization probes to identify Th2-specific-encoding nucleic acids (e.g., Th2-specific mRNA) and fragments for use as PCR primers for the amplification or mutation of Th2-specific nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

Nucleotide sequences encoding the Th2-specific proteins of the present invention include sequences set forth in SEQ ID NOs: 1, 3, 5, 7, 9, 11, 13, and 15, the nucleotide sequences of the cDNA inserts of the plasmids deposited with the ATCC as Accession Numbers 203302, 203569, and 203797 (referred to as the “cDNA of ATCC 203302,” the “cDNA of ATCC 203569,” or the “cDNA of ATCC 203797”), and complements thereof By “complement” is intended a nucleotide sequence that is sufficiently complementary to a given nucleotide sequence such that it can hybridize to the given nucleotide sequence to thereby form a stable duplex. The corresponding amino acid sequences for the Th2-specific proteins encoded by these nucleotide sequences are set forth in SEQ ID NOs: 2, 4, 6, 8, 10, 12, 14, and 16, respectively.

Nucleic acid molecules that are fragments of these Th2-specific nucleotide sequences are also encompassed by the present invention. By “fragment” is intended a portion of the nucleotide sequence encoding a Th2-specific protein of the invention. A fragment of a Th2-specific nucleotide sequence may encode a biologically active portion of a Th2-specific protein, or it may be a fragment that can be used as a hybridization probe or PCR primer using methods disclosed below. A biologically active portion of a Th2-specific protein can be prepared by isolating a portion of one of the Th2-specific nucleotide sequences of the invention, expressing the encoded portion of the Th2-specific protein (e.g., by recombinant expression in vitro), and assessing the activity of the encoded portion of the Th2-specific protein. Generally, nucleic acid molecules that are fragments of a Th2-specific nucleotide sequence comprise at least 15, 20, 50, 75, 100, 325, 350, 375, 400, 425, 450, 500, 550, 600, 650, 700, 800, 900, 1,000, 1,100, 1,200, 1,300, 1350,1400, 1450, 1500, or 1550 nucleotides, or up to the number of nucleotides present in a full-length Th2-specific nucleotide sequence disclosed herein (for example, 3631, 1587, 4382, 2080, 3266, 2703, 1795, or 3868 nucleotides for SEQ ID NO: 1, 3, 5, 7, 9, 11, 13, or 15, respectively) depending upon the intended use.

It is understood that isolated fragments include any contiguous sequence not disclosed prior to the invention as well as sequences that are substantially the same and which are not disclosed. Accordingly, if a fragment is disclosed prior to the present invention, that fragment is not intended to be encompassed by the invention. When a sequence is not disclosed prior to the present invention, an isolated nucleic acid fragment is at least about 12, 15, 20, 25, or 30 contiguous nucleotides. Other regions of the nucleotide sequence may comprise fragments of various sizes, depending upon potential homology with previously disclosed sequences.

For h1022, for example, nucleotide sequences 1 to about 210 and about 565 to about 602 are not disclosed prior to the present invention. The nucleotide sequence from about 1 to about 325 encompasses fragments greater than 20, 21, or 25 nucleotides; the nucleotide sequence from about 306 to about 602 encompasses fragments greater than about 148, 150, or 160 nucleotides; the nucleotide sequence from about 600 to about 850 encompasses fragments greater than about 212, 215, or 220 nucleotides; the nucleotide sequence from about 815 to about 1006 encompasses fragments greater than about 54, 58, 60, or 70 nucleotides; the nucleotide sequence from about 1006 to about 1281 encompasses fragments greater than about 32, 35, or 40 nucleotides; and the nucleotide sequence from about 1200 to about 1795 encompasses fragments greater than about 490 or 500 nucleotides.

For h1419, for example, nucleotide sequences 1 to about 1274, about 1385 to about 2146, about 2190 to about 2202, about 2404 to about 2434, about 2563 to about 2678, and about 3425 to about 3790 are not disclosed prior to the present invention. The nucleotide sequence from about 1 to about 2146 encompasses fragments greater than about 17, 20, or 25 nucleotides; the nucleotide sequence from about 2140 to about 2806 encompasses fragments greater than about 21, 23, or 25 nucleotides; the nucleotide sequence from about 2806 to about 3406 encompasses fragments greater than about 263, 265, or 275 nucleotides; and the nucleotide sequence from about 3406 to about 3868 encompasses fragments greater than about 37, 38, 40, or 45 nucleotides.

A fragment of a Th2-specific nucleotide sequence that encodes a biologically active portion of a Th2-specific protein of the invention will encode at least 15, 25, 30, 50, 100, 150, 200, 250, 300, 350, 400, or 450 contiguous amino acids, or up to the total number of amino acids present in a full-length Th2-specific protein of the invention (for example, 392, 464, 200, 198, 481, or 384 amino acids for SEQ ID NO: 2, 4 and 6, 8 and 10, 12, 14, or 16, respectively). Fragments of a Th2-specific nucleotide sequence that are useful as hybridization probes for PCR primers generally need not encode a biologically active portion of a Th2-specific protein.

Nucleic acid molecules that are variants of the Th2-specific nucleotide sequences disclosed herein are also encompassed by the present invention. “Variants” of the Th2-specific nucleotide sequences include those sequences that encode the Th2-specific proteins disclosed herein but that differ conservatively because of the degeneracy of the genetic code. These naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the Th2-specific proteins disclosed in the present invention as discussed below. Generally, nucleotide sequence variants of the invention will have at least 45%, 55%, 65%, 75%, 85%, 95%, or 98% identity to the nucleotide sequences disclosed herein. A variant Th2-specific nucleotide sequence will encode a Th2-specific protein that has an amino acid sequence having at least 45%, 55%, 65%, 75%, 85%, 95%, or 98% identity to an amino acid sequence of a Th2-specific protein disclosed herein.

In addition to the Th2-specific nucleotide sequences shown in SEQ ID NOs: 1, 3, 5, 7, 9, 11, 13, and 15, the nucleotide sequence of the cDNA of ATCC 203302, the nucleotide sequence of the cDNA of ATCC 230569, and the nucleotide sequence of the cDNA of ATCC 203797, it will be appreciated by those skilled in the art that DNA sequence polymorphisms that lead to changes in the amino acid sequences of Th2-specific proteins may exist within a population (e.g., the human population). Such genetic polymorphism in a Th2-specific gene may exist among individuals within a population due to natural allelic variation. An allele is one of a group of genes that occur alternatively at a given genetic locus. As used herein, the terms “gene” and “recombinant gene” refer to nucleic acid molecules comprising an open reading frame encoding a Th2-specific protein, preferably a mammalian Th2-specific protein. As used herein, the phrase “allelic variant” refers to a nucleotide sequence that occurs at a Th2-specific locus or to a polypeptide encoded by the nucleotide sequence. Such natural allelic variations can typically result in 1-5% variance in the nucleotide sequence of the Th2-specific gene. Any and all such nucleotide variations and resulting amino acid polymorphisms or variations in a Th2-specific sequence that are the result of natural allelic variation and that do not alter the functional activity of Th2-specific proteins are intended to be within the scope of the invention.

Moreover, nucleic acid molecules encoding Th2-specific proteins from other species (Th2-specific homologues), which have a nucleotide sequence differing from that of the Th2-specific sequences disclosed herein, are intended to be within the scope of the invention. Nucleic acid molecules corresponding to natural allelic variants and homologues of the Th2-specific cDNAs of the invention can be isolated based on their identity to the mouse Th2-specific nucleic acids disclosed herein using the mouse cDNAs, or a portion thereof, as a hybridization probe according to standard hybridization techniques under stringent hybridization conditions as disclosed below.

In addition to naturally-occurring allelic variants of the Th2-specific sequence that may exist in the population, the skilled artisan will further appreciate that changes can be introduced by mutation into the nucleotide sequences of the invention thereby leading to changes in the amino acid sequence of the encoded Th2-specific protein, without altering the biological activity of the Th2-specific protein. Thus, an isolated nucleic acid molecule encoding a Th2-specific protein having a sequence that differs from that of SEQ ID NO: 2, 4, 6, 8, 10, 12, 14, or 16 can be created by introducing one or more nucleotide substitutions, additions, or deletions into the nucleotide sequences disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, preferably, conservative amino acid substitutions may be made at one-or more predicted, preferably nonessential amino acid residues. A “nonessential” amino acid residue is a residue that can be altered from the wild-type sequence of a Th2-specific protein (e.g., the sequence of SEQ ID NO: 2, 4, 6, 8, 10, 12, 14, or 16) without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, such as the cysteine residues of clone 1228, or for amino acid residues residing within a conserved motif, such as the PPP motif (present as FDPPPF, aa 113-118 of SEQ ID NO: 12) and YMFM motif (aa 178-181 of SEQ ID NO: 12) of 1228, where such residues are essential for protein activity.

Alternatively, variant Th2-specific nucleotide sequences can be made by introducing mutations randomly along all or part of a Th2-specific coding sequence, such as by saturation mutagenesis, and the resultant mutants can be screened for Th2-specific biological activity to identify mutants that retain activity. Following mutagenesis, the encoded protein can be expressed recombinantly, and the activity of the protein can be determined using standard assay techniques.

Thus the nucleotide sequences of the invention include those sequences disclosed herein as well as fragments and variants thereof The Th2-specific nucleotide sequences of the invention, and fragments and variants thereof, can be used as probes and/or primers to identify and/or clone Th2-specific homologues in other cell types, e.g., from other tissues, as well as Th2-specific homologues from other mammals. Such probes can be used to detect transcripts or genomic sequences encoding the same or identical proteins. These probes can be used as part of a diagnostic test kit for identifying cells or tissues that misexpress a Th2-specific protein, such as by measuring levels of a Th2-specific-encoding nucleic acid in a sample of cells from a subject, e.g., detecting Th2-specific mRNA levels or determining whether a genomic Th2-specific gene has been mutated or deleted.

In this manner, methods such as PCR, hybridization, and the like can be used to identify such sequences having substantial identity to the sequences of the invention. See, for example, Sambrook et al. (1989) Molecular Cloning: Laboratory Manual (2d ed., Cold Spring Harbor Laboratory Press, Plainview, N.Y.) and Innis, et al. (1990) PCR Protocols: A Guide to Methods and Applications (Academic Press, NY). Th2-specific nucleotide sequences isolated based on their sequence identity to the Th2-specific nucleotide sequences set forth herein or to fragments and variants thereof are encompassed by the present invention.

In a hybridization method, all or part of a known Th2-specific nucleotide sequence can be used to screen cDNA or genomic libraries. Methods for construction of such cDNA and genomic libraries are generally known in the art and are disclosed in Sambrook et al. (1989) Molecular Cloning: A Laboratory Manual (2d ed., Coldspring Harbor Laboratory Press, Plainview, N.Y.). The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known Th2-specific nucleotide sequences disclosed herein. Degenerate primers designed on the basis of conserved nucleotides or amino acid residues in a known Th2-specific nucleotide sequence or encoded amino acid sequence can additionally be used. The probe typically comprises a region of nucleotide sequence that hybridizes under stringent conditions to at least about 12, preferably about 25, more preferably about 50, 75, 100, 125, 150, 175, 200, 250, 300, 350, or 400 consecutive nucleotides of a Th2-specific nucleotide sequence of the invention or a fragment or variant thereof. Preparation of probes for hybridization is generally known in the art and is disclosed in Sambrook et al. (1989) Molecular Cloning: A Laboratory Manual (2d ed., Coldspring Harbor Laboratory Press, Plainview, N.Y.), herein incorporated by reference.

For example, in one embodiment, a previously unidentified Th2-specific nucleic acid molecule hybridizes under stringent conditions to a probe that is a nucleic acid molecule comprising one of the Th2-specific nucleotide sequences of the invention or a fragment thereof In another embodiment, the previously unknown Th2-specific nucleic acid molecule is at least 300, 325, 350, 375, 400, 425, 450, 500, 550, 600, 650, 700, 800, 900, 1000, 2,000, 3,000, 4,000 or 5,000 nucleotides in length and hybridizes under stringent conditions to a probe that is a nucleic acid molecule comprising one of the Th2-specific nucleotide sequences disclosed herein or a fragment thereof

Accordingly, in another embodiment, an isolated previously unknown Th2-specific nucleic acid molecule of the invention is at least 300, 325, 350, 375, 400, 425, 450, 500, 550, 600, 650, 700, 800, 900, 1000, 1,100, 1,200, 1,300, or 1,400 nucleotides in length and hybridizes under stringent conditions to a probe that is a nucleic acid molecule comprising one of the nucleotide sequences of the invention, preferably the coding sequence set forth in SEQ ID NO: 1, 3, 5, 7, 9, 11, 13, or 15, the cDNA of ATCC 203302, the cDNA of ATCC 203569, the cDNA of ATCC 203797, or a complement, fragment, or variant thereof.

As used herein, the term “hybridizes under stringent conditions” is intended to describe conditions for hybridization and washing under which nucleotide sequences having at least 60%, 65%, 70%, preferably 75% identity to each other typically remain hybridized to each other. Such stringent conditions are known to those skilled in the art and can be found in Current Protocols in Molecular Biology (John Wiley & Sons, New York (1989)), 6.3.1-6.3.6. A preferred, non-limiting example of stringent hybridization conditions is hybridization in 6× sodium chloride/sodium citrate (SSC) at about 45° C., followed by one or more washes in 0.2×SSC, 0.1% SDS at 50-65° C. In another preferred embodiment, stringent conditions comprise hybridization in 6×SSC at 42° C., followed by washing with 1×SSC at 55° C. Preferably, an isolated nucleic acid molecule that hybridizes under stringent conditions to a Th2-specific sequence of the invention corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

Thus, in addition to the Th2-specific nucleotide sequences disclosed herein and fragments and variants thereof, the isolated nucleic acid molecules of the invention also encompass homologous DNA sequences identified and isolated from other cells and/or organisms by hybridization with entire or partial sequences obtained from the Th2-specific nucleotide sequences disclosed herein or variants and fragments thereof

The present invention also encompasses antisense nucleic acid molecules, i.e., molecules that are complementary to a sense nucleic acid encoding a protein, e.g., complementary to the coding strand of a double-stranded cDNA molecule, or complementary to an mRNA sequence. Accordingly, an antisense nucleic acid can hydrogen bond to a sense nucleic acid. The antisense nucleic acid can be complementary to an entire Th2-specific coding strand, or to only a portion thereof, e.g., all or part of the protein coding region (or open reading frame). An antisense nucleic acid molecule can be antisense to a noncoding region of the coding strand of a nucleotide sequence encoding a Th2-specific protein. The noncoding regions are the 5′ and 3′ sequences that flank the coding region and are not translated into amino acids.

Given the coding-strand sequences encoding a Th2-specific protein disclosed herein (e.g., SEQ ID NOs: 1, 3, 5, 7, 9, 11, 13, and 15), antisense nucleic acids of the invention can be designed according to the rules of Watson and Crick base pairing. The antisense nucleic acid molecule can be complementary to the entire coding region of Th2-specific mRNA, but more preferably is an oligonucleotide that is antisense to only a portion of the coding or noncoding region of Th2-specific mRNA. For example, the antisense oligonucleotide can be complementary to the region surrounding the translation start site of Th2-specific mRNA. An antisense oligonucleotide can be, for example, about 5, 10, 15, 20, 25, 30, 35, 40, 45, or 50 nucleotides in length. An antisense nucleic acid of the invention can be constructed using chemical synthesis and enzymatic ligation procedures known in the art.

For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, including, but not limited to, for example e.g., phosphorothioate derivatives and acridine substituted nucleotides. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a Th2-specific protein to thereby inhibit expression of the protein, e.g., by inhibiting transcription and/or translation. An example of a route of administration of antisense nucleic acid molecules of the invention includes direct injection at a tissue site. Alternatively, antisense nucleic acid molecules can be modified to target selected cells and then administered systemically. For example, antisense molecules can be linked to peptides or antibodies to form a complex that specifically binds to receptors or antigens expressed on a selected cell surface. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes, which are catalytic RNA molecules with ribonuclease activity that are capable of cleaving a single-stranded nucleic acid, such as an mRNA, to which they have a complementary region. Ribozymes (e.g., hammerhead ribozymes (described in Haselhoff and Gerlach (1988) Nature 334:585-591)) can be used to catalytically cleave Th2-specific mRNA transcripts to thereby inhibit translation of Th2-specific mRNA. A ribozyme having specificity for a Th2-specific-encoding nucleic acid can be designed based upon the nucleotide sequence of a Th2-specific cDNA disclosed herein (e.g., SEQ ID NO: 1, 3, 5, 7, 9, 11, 13, or 15). See, e.g., Cech et al., U.S. Pat. No. 4,987,071; and Cech et al., U.S. Pat. No. 5,116,742. Alternatively, Th2-specific mRNA can be used to select a catalytic RNA having a specific ribonuclease activity from a pool of RNA molecules. See, e.g., Bartel and Szostak (1993) Science 261:1411-1418.

The invention also encompasses nucleic acid molecules that form triple helical structures. For example, Th2-specific gene expression can be inhibited by targeting nucleotide sequences complementary to the regulatory region of the Th2-specific protein (e.g., the Th2-specific promoter and/or enhancers) to form triple helical structures that prevent transcription of the Th2-specific gene in target cells. See generally Helene (1991) Anticancer Drug Des. 6(6):569; Helene (1992) Ann. N.Y. Acad. Sci. 660:27; and Maher (1992) Bioassays 14(12):807.

In preferred embodiments, the nucleic acid molecules of the invention can be modified at the base moiety, sugar moiety, or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid-phase peptide synthesis protocols as described in Hyrup et al. (1996), supra; Perry-O'Keefe et al. (1996) Proc. Natl. Acad. Sci. USA 93:14670.

PNAs of a Th2-specific molecule can be used in therapeutic and diagnostic applications. For example, PNAs can be used as antisense or antigene agents for sequence-specific modulation of gene expression by, e.g., inducing transcription or translation arrest or inhibiting replication. PNAs of the invention can also be used, e.g., in the analysis of single base pair mutations in a gene by, e.g., PNA-directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996), supra).

In another embodiment, PNAs of a Th2-specific molecule can be modified, e.g., to enhance their stability, specificity, or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra; Finn et al. (1996) Nucleic Acids Res. 24(17):3357-63; Mag et al. (1989) Nucleic Acids Res. 17:5973; and Peterser et al. (1975) Bioorganic Med. Chem. Lett. 5:1119.

II. Isolated Th2-Specific Proteins and Anti-Th2-Specific Antibodies

Th2-specific proteins are also encompassed within the present invention. By “Th2-specific protein” is intended proteins having the amino acid sequence set forth in SEQ ID NO: 2, 4, 6, 8, 10, 12, 14, or 16, as well as fragments, biologically active portions, and variants thereof

“Fragments” or “biologically active portions” include polypeptide fragments suitable for use as immunogens to raise anti-Th2-specific antibodies. Fragments include peptides comprising amino acid sequences sufficiently identical to or derived from the amino acid sequences of a Th2-specific protein of the invention and exhibiting at least one activity of a Th2-specific protein, but which include fewer amino acids than the full-length Th2-specific proteins disclosed herein. Typically, biologically active portions comprise a domain or motif with at least one activity of the Th2-specific protein. A biologically active portion of a Th2-specific protein can be a polypeptide that is, for example, 10, 25, 50, 100 or more amino acids in length. Such biologically active portions can be prepared by recombinant techniques and evaluated for one or more of the functional activities of a native Th2-specific protein.

By “variants” is intended proteins or polypeptides having an amino acid sequence that is at least about 45%, 55%, 65%, preferably about 75%, 85%, 95%, or 98% identical to the amino acid sequence of SEQ ID NO: 2, 4, 6, 8, 10, 12, 14, or 16. Variants also include polypeptides encoded by the cDNA insert of the plasmid deposited with ATCC as Accession Number 203302, ATCC Accession Number 203569, or ATCC Accession Number 203797, or polypeptides encoded by a nucleic acid molecule that hybridizes to a nucleic acid molecule of SEQ ID NO: 1, 3, 5, 7, 9, 11, 13, or 15, or a complement thereof, under stringent conditions. Such variants generally retain the functional activity of the Th2-specific proteins of the invention. Variants include polypeptides that differ in amino acid sequence due to natural allelic variation or mutagenesis.

The invention also provides Th2-specific chimeric or fusion proteins. As used herein, a Th2-specific “chimeric protein” or “fusion protein” comprises a Th2-specific polypeptide operably linked to a non-Th2-specific polypeptide. A “Th2-specific polypeptide” refers to a polypeptide having an amino acid sequence corresponding to a Th2-specific protein, whereas a “non-Th2-specific polypeptide” refers to a polypeptide having an amino acid sequence corresponding to a protein that is not substantially identical to the Th2-specific protein, e.g., a protein that is different from the Th2-specific protein and which is derived from the same or a different organism. Within a Th2-specific fusion protein, the Th2-specific polypeptide can correspond to all or a portion of a Th2-specific protein, preferably at least one biologically active portion of a Th2-specific protein. Within the fusion protein, the term “operably linked” is intended to indicate that the Th2-specific polypeptide and the non-Th2-specific polypeptide are fused in-frame to each other. The non-Th2-specific polypeptide can be fused to the N-terminus or C-terminus of the Th2-specific polypeptide.

One useful fusion protein is a GST-Th2-specific fusion protein in which the Th2-specific sequences are fused to the C-terminus of the GST sequences. Such fusion proteins can facilitate the purification of recombinant Th2-specific proteins.

In yet another embodiment, the fusion protein is a Th2-specific-immunoglobulin fusion protein in which all or part of a Th2-specific protein is fused to sequences derived from a member of the immunoglobulin protein family. The Th2-specific-immunoglobulin fusion proteins of the invention can be incorporated into pharmaceutical compositions and administered to a subject to inhibit an interaction between a Th2-specific ligand and a Th2-specific protein on the surface of a cell, thereby suppressing Th2-specific-mediated signal transduction in vivo. The Th2-specific-immunoglobulin fusion proteins can be used to affect the bioavailability of a Th2-specific cognate ligand. Inhibition of the Th2-specific ligand/Th2-specific interaction may be useful therapeutically, both for treating proliferative and differentiative disorders and for modulating (e.g., promoting or inhibiting) cell survival. Moreover, the Th2-specific-immunoglobulin fusion proteins of the invention can be used as immunogens to produce anti-Th2-specific antibodies in a subject, to purify Th2-specific ligands, and in screening assays to identify molecules that inhibit the interaction of a Th2-specific protein with a Th2-specific ligand.

Preferably, a Th2-specific chimeric or fusion protein of the invention is produced by standard recombinant DNA techniques. For example, DNA fragments coding for the different polypeptide sequences may be ligated together in-frame, or the fusion gene can be synthesized, such as with automated DNA synthesizers. Alternatively, PCR amplification of gene fragments can be carried out using anchor primers that give rise to complementary overhangs between two consecutive gene fragments, which can subsequently be annealed and reamplified to generate a chimeric gene sequence (see, e.g., Ausubel et al., eds. (1995) Current Protocols in Molecular Biology) (Greene Publishing and Wiley-Interscience, NY). Moreover, a Th2-specific-encoding nucleic acid can be cloned into a commercially available expression vector such that it is linked in-frame to an existing fusion moiety.

Variants of the Th2-specific proteins can function as either Th2-specific agonists (mimetics) or as Th2-specific antagonists. Variants of the Th2-specific protein can be generated by mutagenesis, e.g., discrete point mutation or truncation of the Th2-specific protein. An agonist of the Th2-specific protein can retain substantially the same, or a subset, of the biological activities of the naturally occurring form of the Th2-specific protein. An antagonist of the Th2-specific protein can inhibit one or more of the activities of the naturally occurring form of the Th2-specific protein by, for example, competitively binding to a downstream or upstream member of a cellular signaling cascade that includes the Th2-specific protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the Th2-specific proteins.

Variants of the Th2-specific protein that function as either Th2-specific agonists or as Th2-specific antagonists can be identified by screening combinatorial libraries of mutants, e.g., truncation mutants, of the Th2-specific protein for Th2-specific protein agonist or antagonist activity. In one embodiment, a variegated library of Th2-specific variants is generated by combinatorial mutagenesis at the nucleic acid level and is encoded by a variegated gene library. A variegated library of Th2-specific variants can be produced by, for example, enzymatically ligating a mixture of synthetic oligonucleotides into gene sequences such that a degenerate set of potential Th2-specific sequences is expressible as individual polypeptides, or alternatively, as a set of larger fusion proteins (e.g., for phage display) containing the set of Th2-specific sequences therein. There are a variety of methods that can be used to produce libraries of potential Th2-specific variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential Th2-specific sequences. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the Th2-specific protein coding sequence can be used to generate a variegated population of Th2-specific fragments for screening and subsequent selection of variants of a Th2-specific protein. In one embodiment, a library of coding sequence fragments can be generated by treating a double-stranded PCR fragment of a Th2-specific coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, one can derive an expression library that encodes N-terminal and internal fragments of various sizes of the Th2-specific protein.

Several techniques are known in the art for screening gene products of combinatorial libraries made by point mutations or truncation and for screening cDNA libraries for gene products having a selected property. Such techniques are adaptable for rapid screening of the gene libraries generated by the combinatorial mutagenesis of Th2-specific proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify Th2-specific variants (Arkin and Yourvan (1992) Proc. Natl. Acad. Sci. USA 89:7811-7815; Delgrave et al. (1993) Protein Engineering 6(3):327-331).

An isolated Th2-specific polypeptide of the invention can be used as an immunogen to generate corresponding antibodies that bind Th2-specific proteins using standard techniques for polyclonal and monoclonal antibody preparation By “corresponding antibody” is intended that the antibody binds the particular Th2-specific protein used as the immunogen. The full-length Th2-specific protein can be used or, alternatively, the invention provides antigenic peptide fragments of Th2-specific proteins for use as immunogens. The antigenic peptide of a Th2-specific protein comprises at least 8, preferably 10, 15, 20, or 30 amino acid residues of the amino acid sequence shown in SEQ ID NO: 2, 4, 6, 8, 10, 12, 14, or 16 and encompasses an epitope of a Th2-specific protein such that an antibody raised against the peptide forms a specific immune complex with the Th2-specific protein. Preferred epitopes encompassed by the antigenic peptide are regions of a Th2-specific protein that are located on the surface of the protein, e.g., hydrophilic regions.

Accordingly, another aspect of the invention pertains to anti-Th2-specific polyclonal and monoclonal antibodies that bind a Th2-specific protein. Polyclonal anti-Th2-specific antibodies can be prepared by immunizing a suitable subject (e.g., rabbit, goat, mouse, or other mammal) with a Th2-specific immunogen. The anti-Th2-specific antibody titer in the immunized subject can be monitored over time by standard techniques, such as with an enzyme linked immunosorbent assay (ELISA) using immobilized Th2-specific protein. At an appropriate time after immunization, e.g., when the anti-Th2-specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:550-52; Kenneth (1980) in Monoclonal Antibodies: A New Dimension In Biological Analyses (Plenum Publishing Corp., NY; and Lerner (1981) Yale J. Biol. Med, 54:387-402).

Alternative to preparing monoclonal antibody-secreting hybridomas, a monoclonal anti-Th2-specific antibody can be identified and isolated by screening a recombinant combinatorial immunoglobulin library (e.g., an antibody phage display library) with a Th2-specific protein to thereby isolate immunoglobulin library members that bind the Th2-specific protein. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant anti-Th2-specific antibodies, such as chimeric and humanized monoclonal antibodies, comprising both human and nonhuman portions, which can be made using standard recombinant DNA techniques, are within the scope of the invention. Such chimeric and humanized monoclonal antibodies can be produced by recombinant DNA techniques known in the art, for example using methods described in PCT Publication Nos. WO 86101533 and WO 87/02671; European Patent Application Nos. 184,187, 171,496, 125,023, and 173,494; U.S. Pat. Nos. 4,816,567 and 5,225,539; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) Bio/Techniques 4:214; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

An anti-Th2-specific antibody (e.g., monoclonal antibody) can be used to isolate Th2-specific proteins by standard techniques, such as affinity chromatography or immunoprecipitation. An anti-Th2-specific antibody can facilitate the purification of natural Th2-specific protein from cells and of recombinantly produced Th2-specific protein expressed in host cells. Moreover, an anti-Th2-specific antibody can be used to detect Th2-specific protein (e.g., in a cellular lysate or cell supernatant) in order to evaluate the abundance and pattern of expression of the Th2-specific protein. Anti-Th2-specific antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent-materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84:Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The antibody-associated molecules of the invention, i.e., corresponding antibodies that bind Th2-specific proteins and such corresponding antibodies that are conjugated to a therapeutic moiety, can be used alone or in any combination, with or without additional therapeutic moieties, to modify a given biological response, e.g., modulating a Th2 response, in accordance with methods of the invention. Thus, for example, modulation of a given biological response is achieved by administering to a subject antibody-associated molecules of the invention, including, but not limited to, administering 1) at least one antibody of the invention, 2) at least one antibody of the invention and at least one free (i.e., not conjugated to an antibody) therapeutic moiety, 3) at least one antibody of the invention that is conjugated to a therapeutic moiety, 4) at least one antibody of the invention and at least one antibody of the invention that is conjugated to a therapeutic moiety, 5) at least one antibody of the invention that is conjugated to a therapeutic moiety and at least one free therapeutic moiety, or 6) at least one antibody of the invention, at least one antibody of the invention that is conjugated to a therapeutic moiety, and at least one free therapeutic moiety. These antibody-associated molecules may be administered to the subject separately from one another, either at the same time or at a different time. Alternatively, these antibody-associated molecules may be administered at the same time, as in a mixture, for example, a mixture comprising both an antibody of the invention and an antibody that is conjugated to a therapeutic moiety, or a mixture comprising any combination of the antibody-associated molecules of the invention, as noted above.

III. Recombinant Expression Vectors and Host Cells

Another aspect of the invention pertains to vectors, preferably expression vectors, containing a nucleic acid encoding a Th2-specific protein (or a portion thereof). “Vector” refers to a nucleic acid molecule capable of transporting another nucleic acid to which it has been linked, such as a “plasmid”, a circular double-stranded DNA loop into which additional DNA segments can be ligated, or a viral vector, where additional DNA segments can be ligated into the viral genome. The vectors are useful for autonomous replication in a host cell or may be integrated into the genome of a host cell upon introduction into the host cell, and thereby are replicated along with the host genome (e.g., nonepisomal mammalian vectors). Expression vectors are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses, and adeno-associated viruses), that serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, operably linked to the nucleic acid sequence to be expressed. “Operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., Th2-specific proteins, mutant forms of Th2-specific proteins, fusion proteins, etc.).

The recombinant expression vectors of the invention can be designed for expression of Th2-specific protein in prokaryotic or eukaryotic host cells. Expression of proteins in prokaryotes is most often carried out in E. coli with vectors containing constitutive or inducible promoters directing the expression of either fusion or nonfusion proteins. Fusion vectors add a number of amino acids to a protein encoded therein, usually to the amino terminus of the recombinant protein. Typical fusion expression vectors include pGEX (Pharmacia Biotech Inc; Smith and Johnson (1988) Gene 67:31-40), pMAL (New England Biolabs, Beverly, Mass.), and pRIT5 (Pharmacia, Piscataway, N.J.) which fuse glutathione S-transferase (GST), maltose E binding protein, or protein A, respectively, to the target recombinant protein. Examples of suitable inducible nonfusion E. coli expression vectors include pTrc (Amann et al. (1988) Gene 69:301-315) and pET 11d (Studier et al. (1990) in Gene Expression Technology: Methods in Enzymology 185 (Academic Press, San Diego, Calif.), pp. 60-89). Strategies to maximize recombinant protein expression in E. coli can be found in Gottesman (1990) in Gene Expression Technology: Methods in Enzymology 185 (Academic Press, Calif.), pp. 119-128 and Wada et al. (1992) Nucleic Acids Res. 20:2111-2118. Target gene expression from the pTrc vector relies on host RNA polymerase transcription from a hybrid trp-lac fusion promoter.

Suitable eukaryotic host cells include insect cells (examples of Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cereivisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)). Suitable mammalian cells include Chinese hamster ovary cells (CHO) or COS cells. In mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see chapters 16 and 17 of Sambrook et al. (1989) Molecular Cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.). Alternatively, the recombinant expression vector can be transcribed and translated in vitro, for example using T7 promoter regulatory sequences and T7 polymerase.

The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell but are still included within the scope of the term as used herein.

In one embodiment, the expression vector is a recombinant mammalian expression vector that comprises tissue-specific regulatory elements that direct expression of the nucleic acid preferentially in a particular cell type. Suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Patent Publication No. 264,166). Developmentally-regulated promoters are also encompassed, for example the murine hox promoters (Kessel and Gruss (1990) Science 249:374-379), the α-fetoprotein promoter (Campes and Tilghman (1989) Genes Dev. 3:537-546), and the like.

The invention further provides a recombinant expression vector comprising a DNA molecule of the invention cloned into the expression vector in an antisense orientation. That is, the DNA molecule is operably linked to a regulatory sequence in a manner that allows for expression (by transcription of the DNA molecule) of an RNA molecule that is antisense to Th2-specific mRNA. Regulatory sequences operably linked to a nucleic acid cloned in the antisense orientation can be chosen to direct the continuous expression of the antisense RNA molecule in a variety of cell types, for instance viral promoters and/or enhancers, or regulatory sequences can be chosen to direct constitutive, tissue-specific, or cell-type-specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (1986) Reviews—Trends in Genetics, Vol. 1(1).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (1989) Molecular Cloning: A Laboraty Manual (2d ed., Cold Spring Harbor Laboratory Press, Plainview, N.Y.) and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin, and methotrexate. Nucleic acid encoding a selectable marker can be introduced into a host cell on the same vector as that encoding a Th2-specific protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) Th2-specific protein. Accordingly, the invention further provides methods for producing Th2-specific protein using the host cells of the invention. In one embodiment, the method comprises culturing the host cell of the invention, into which a recombinant expression vector encoding a Th2-specific protein has been introduced, in a suitable medium such that Th2-specific protein is produced. In another embodiment, the method further comprises isolating Th2-specific protein from the medium or the host cell.

The host cells of the invention can also be used to produce nonhuman transgenic animals. For example, in one embodiment, a host cell of the invention is a fertilized oocyte or an embryonic stem cell into which Th2-specific-coding sequences have been introduced. Such host cells can then be used to create nonhuman transgenic animals in which exogenous Th2-specific sequences have been introduced into their genome or homologous recombinant animals in which endogenous Th2-specific sequences have been altered. Such animals are useful for studying the function and/or activity of Th2-specific genes and proteins and for identifying and/or evaluating modulators of Th2-specific activity. As used herein, a “transgenic animal” is a nonhuman animal, preferably a mammal, more preferably a rodent such as a rat or mouse, in which one or more of the cells of the animal includes a transgene. Other examples of transgenic animals include nonhuman primates, sheep, dogs, cows, goats, chickens, amphibians, etc. A transgene is exogenous DNA that is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a nonhuman animal, preferably a mammal, more preferably a mouse, in which an endogenous Th2-specific gene has been altered by homologous recombination between the endogenous gene and an exogenous DNA molecule introduced into a cell of the animal, e.g., an embryonic cell of the animal, prior to development of the animal.

A transgenic animal of the invention can be created by introducing Th2-specific-encoding nucleic acid into the male pronuclei of a fertilized oocyte, e.g., by microinjection, retroviral infection, and allowing the oocyte to develop in a pseudopregnant female foster animal. The Th2-specific cDNA sequence can be introduced as a transgene into the genome of a nonhuman animal. Alternatively, a homologue of the mouse Th2-specific gene can be isolated based on hybridization and used as a transgene. Intronic sequences and polyadenylation signals can also be included in the transgene to increase the efficiency of expression of the transgene. A tissue-specific regulatory sequence(s) can be operably linked to the Th2-specific transgene to direct expression of Th2-specific protein to particular cells. Methods for generating transgenic animals via embryo manipulation and microinjection, particularly animals such as mice, have become conventional in the art and are described, for example, in U.S. Pat. Nos. 4,736,866, 4,870,009, and 4,873,191 and in Hogan (1986) Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the Th2-specific transgene in its genome and/or expression of Th2-specific mRNA in tissues or cells of the animals. A transgenic founder animal can then be used to breed additional animals carrying the transgene. Moreover, transgenic animals carrying a transgene encoding Th2-specific gene can further be bred to other transgenic animals carrying other transgenes.

To create a homologous recombinant animal, one prepares a vector containing at least a portion of a Th2-specific gene or a homologue of the gene into which a deletion, addition, or substitution has been introduced to thereby alter, e.g., functionally disrupt, the Th2-specific gene. In a preferred embodiment, the vector is designed such that, upon homologous recombination, the endogenous Th2-specific gene is functionally disrupted (i.e., no longer encodes a functional protein; also referred to as a “knock out” vector). Alternatively, the vector can be designed such that, upon homologous recombination, the endogenous Th2-specific gene is mutated or otherwise altered but still encodes functional protein (e.g., the upstream regulatory region can be altered to thereby alter the expression of the endogenous Th2-specific protein). In the homologous recombination vector, the altered portion of the Th2-specific gene is flanked at its 5′ and 3′ ends by additional nucleic acid of the Th2-specific gene to allow for homologous recombination to occur between the exogenous Th2-specific gene carried by the vector and an endogenous Th2-specific gene in an embryonic stem cell. The additional flanking Th2-specific nucleic acid is of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation), and cells in which the introduced Th2-specific gene has homologously recombined with the endogenous Th2-specific gene are selected (see, e.g., Li et al. (1992) Cell 69:915). The selected cells are then injected into a blastocyst of an animal (e.g., a mouse) to form aggregation chimeras (see, e.g., Bradley (1987) in Teratocarcinomas and Embryonic Stem Cells: A Practical Approach, ed. Robertson (IRL, Oxford), pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic nonhuman animals containing selected systems that allow for regulated expression of the transgene can be produced. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the nonhuman transgenic animals described herein can also be produced according to the methods described in Wilmut et al. (1997) Nature 385:810-813 and PCT Publication Nos. WO 97/07668 and WO 97/07669.

IV. Pharmaceutical Compositions

The Th2-specific nucleic acid molecules, Th2-specific proteins, and anti-Th2-specific antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compositions of the invention are useful to treat any of the disorders discussed herein. The compositions are provided in therapeutically effective amounts. By “therapeutically effective amount” is intended an amount sufficient to modulate or bring about the desired response. For example, where a Th2 response is to be modulated, a therapeutically effective amount of a composition of the invention would be an amount sufficient to modulate a Th2 response. As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a Th2-specific protein or anti-Th2-specific antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth, or gelatin, an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressurized container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. Depending on the type and severity of the disease, about 1 μg/kg to about 15 mg/kg (e.g., 0.1 to 20 mg/kg) of antibody is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: (a) screening assays; (b) detection assays (e.g., chromosomal mapping, tissue typing, forensic biology); (c) predictive medicine (e.g., diagnostic assays, prognostic assays, monitoring clinical trials, and pharmacogenomics); and (d) methods of treatment (e.g., therapeutic and prophylactic). The isolated nucleic acid molecules of the invention can be used to express Th2-specific protein (e.g., via a recombinant expression vector in a host cell in gene therapy applications), to detect Th2-specific mRNA (e.g., in a biological sample) or a genetic lesion in a Th2-specific gene, and to modulate Th2-specific activity. In addition, the Th2-specific proteins can be used to screen drugs or compounds that modulate the immune response as well as to treat disorders characterized by insufficient or excessive production of Th2-specific protein or production of Th2-specific protein forms that have decreased or aberrant activity compared to Th2-specific wild type protein. In addition, the anti-Th2-specific antibodies of the invention can be used to detect and isolate Th2-specific proteins and modulate Th2-specific activity.

A. Screening Assays

The invention provides a method (also referred to herein as a “screening assay”) for identifying modulators, i.e., candidate or test compounds or agents (e.g., peptides, peptidomimetics, small molecules, or other drugs) that bind to Th2-specific proteins or have a stimulatory or inhibitory effect on, for example, Th2-specific expression or Th2-specific activity.

The test compounds of the present invention can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12: 145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404406; Cwirla et al. (1990) Proc. Natl. Acad. Sci. USA 87:6378-6382; and Felici (1991) J. Mol. Biol. 222:301-310).

Determining the ability of the test compound to bind to the Th2-specific protein can be accomplished, for example, by coupling the test compound with a radioisotope or enzymatic label such that binding of the test compound to the Th2-specific protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In a similar manner, one may determine the ability of the Th2-specific protein to bind to or interact with a Th2-specific target molecule. By “target molecule” is intended a molecule with which a Th2-specific protein binds or interacts in nature. In a preferred embodiment, the ability of the Th2-specific protein to bind to or interact with a Th2-specific target molecule can be determined by monitoring the activity of the target molecule. For example, the activity of the target molecule can be monitored by detecting induction of a cellular second messenger of the target (e.g., intracellular Ca²⁺, diacylglycerol, IP3, etc.), detecting catalytic/enzymatic activity of the target on an appropriate substrate, detecting the induction of a reporter gene (e.g., a Th2-specific-responsive regulatory element operably linked to a nucleic acid encoding a detectable marker, e.g., luciferase), or detecting a cellular response, for example, cellular differentiation or cell proliferation.

In yet another embodiment, an assay of the present invention is a cell-free assay comprising contacting a Th2-specific protein or biologically active portion thereof with a test compound and determining the ability of the test compound to bind to the Th2-specific protein or biologically active portion thereof Binding of the test compound to the Th2-specific protein can be determined either directly or indirectly as described above. In a preferred embodiment, the assay includes contacting the Th2-specific protein or biologically active portion thereof with a known compound that binds Th2-specific protein to form an assay mixture, contacting the assay mixture with a test compound, and determining the ability of the test compound to preferentially bind to Th2-specific protein or biologically active portion thereof as compared to the known compound.

In another embodiment, an assay is a cell-free assay comprising contacting Th2-specific protein or biologically active portion thereof with a test compound and determining the ability of the test compound to modulate (e.g., stimulate or inhibit) the activity of the Th2-specific protein or biologically active portion thereof. Determining the ability of the test compound to modulate the activity of a Th2-specific protein can be accomplished, for example, by determining the ability of the Th2-specific protein to bind to a Th2-specific target molecule as described above for determining direct binding. In an alternative embodiment, determining the ability of the test compound to modulate the activity of a Th2-specific protein can be accomplished by determining the ability of the Th2-specific protein to further modulate a Th2-specific target molecule. For example, the catalytic/enzymatic activity of the target molecule on an appropriate substrate can be determined as previously described.

In yet another embodiment, the cell-free assay comprises contacting the Th2-specific protein or biologically active portion thereof with a known compound that binds a Th2-specific protein to form an assay mixture, contacting the assay mixture with a test compound, and determining the ability of the test compound to preferentially bind to or modulate the activity of a Th2-specific target molecule.

In the above-mentioned assays, it may be desirable to immobilize either a Th2-specific protein or its target molecule to facilitate separation of complexed from uncomplexed forms of one or both of the proteins, as well as to accommodate automation of the assay. In one embodiment, a fusion protein can be provided that adds a domain that allows one or both of the proteins to be bound to a matrix. For example, glutathione-S-transferase/Th2-specific fusion proteins or glutathione-S-transferase/target fusion proteins can be adsorbed onto glutathione sepharose beads (Sigma Chemical, St. Louis, Mo.) or glutathione-derivatized microtitre plates, which are then combined with the test compound or the test compound and either the nonadsorbed target protein or Th2-specific protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of Th2-specific binding or activity determined using standard techniques.

Other techniques for immobilizing proteins on matrices can also be used in the screening assays of the invention. For example, either Th2-specific protein or its target molecule can be immobilized utilizing conjugation of biotin and streptavidin. Biotinylated Th2-specific molecules or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96-well plates (Pierce Chemicals). Alternatively, antibodies reactive with a Th2-specific protein or target molecules but which do not interfere with binding of the Th2-specific protein to its target molecule can be derivatized to the wells of the plate, and unbound target or Th2-specific protein trapped in the wells by antibody conjugation. Methods for detecting such complexes, in addition to those described above for the GST-immobilized complexes, include immunodetection of complexes using antibodies reactive with the Th2-specific protein or target molecule, as well as enzyme-linked assays that rely on detecting an enzymatic activity associated with the Th2-specific protein or target molecule.

In another embodiment, modulators of Th2-specific expression are identified in a method in which a cell is contacted with a candidate compound and the expression of Th2-specific mRNA or protein in the cell is determined relative to expression of Th2-specific mRNA or protein in a cell in the absence of the candidate compound. When expression is greater (statistically significantly greater) in the presence of the candidate compound than in its absence, the candidate compound is identified as a stimulator of Th2-specific mRNA or protein expression. Alternatively, when expression is less (statistically significantly less) in the presence of the candidate compound than in its absence, the candidate compound is identified as an inhibitor of Th2-specific mRNA or protein expression. The level of Th2-specific mRNA or protein expression in the cells can be determined by methods described herein for detecting Th2-specific mRNA or protein.

In yet another aspect of the invention, the Th2-specific proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with Th2-specific protein (“Th2-specific-binding proteins” or “Th2-specific-bp”) and modulate Th2-specific activity. Such Th2-specific-binding proteins are also likely to be involved in the propagation of signals by the Th2-specific proteins as, for example, upstream or downstream elements of the Th2-specific pathway.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (1) map their respective genes on a chromosome; (2) identify an individual from a minute biological sample (tissue typing); and (3) aid in forensic identification of a biological sample. These applications are described in the subsections below.

1. Chromosome Mapping

The isolated complete or partial Th2-specific gene sequences of the invention can be used to map their respective Th2-specific genes on a chromosome, thereby facilitating the location of gene regions associated with genetic disease. Computer analysis of Th2-specific sequences can be used to rapidly select PCR primers (preferably 15-25 bp in length) that do not span more than one exon in the genomic DNA, thereby simplifying the amplification process. These primers can then be used for PCR screening of somatic cell hybrids containing individual human chromosomes. Only those hybrids containing the human gene corresponding to the Th2-specific sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow (because they lack a particular enzyme), but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes (D'Eustachio et al. (1983) Science 220:919-924). Somatic cell hybrids containing only fragments of human chromosomes can also be produced by using human chromosomes with translocations and deletions.

Other mapping strategies that can similarly be used to map a Th2-specific sequence to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries. Furthermore, fluorescence in situ hybridization (FISH) of a DNA sequence to a metaphase chromosomal spread can be used to provide a precise chromosomal location in one step. For a review of this technique, see Verma eta a. (1988) Human Chromosomes: A Manual of Basic Techniques (Pergamon Press, NY). The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results in a reasonable amount of time.

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with the Th2-specific gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The Th2-specific sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes and probed on a Southern blot to yield unique bands for identification. The sequences of the present invention are useful as additional DNA markers for RFLP (described in U.S. Pat. No. 5,272,057).

Furthermore, the sequences of the present invention can be used to provide an alternative technique for determining the actual base-by-base DNA sequence of selected portions of an individual's genome. Thus, the Th2-specific sequences of the invention can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The Th2-specific sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. The noncoding sequences of SEQ ID NO: 1, 3, 5, 7, 9, 11, 13, or 15 can comfortably provide positive individual identification with a panel of perhaps 10 to 1,000 primers that each yield a noncoding amplified sequence of 100 bases. If predicted coding sequences, such as those in SEQ ID NO: 1, 3 5, 7, 9, 11, 13, or 15 are used, a more appropriate number of primers for positive individual identification would be 500 to 2,000.

3. Use of Partial Th2-Specific Sequences in Forensic Biology

DNA-based identification techniques can also be used in forensic biology. In this manner, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” that is unique to a particular individual. As mentioned above, actual base sequence information can be used for identification as an accurate alternative to patterns formed by restriction enzyme generated fragments. Sequences targeted to noncoding regions of SEQ ID NO: 1, 3, 5, 7, 9, 11, 13, or 15 are particularly appropriate for this use as greater numbers of polymorphisms occur in the noncoding regions, making it easier to differentiate individuals using this technique. Examples of polynucleotide reagents include the Th2-specific sequences or portions thereof, e.g., fragments derived from the noncoding regions of SEQ ID NO: 1, 3, 5, 7, 9, 11, 13, or 15 having a length of at least 20 or 30 bases.

The Th2-specific sequences described herein can further be used to provide polynucleotide reagents, e.g., labeled or labelable probes that can be used in, for example, an in situ hybridization technique, to identify a specific tissue. This can be very useful in cases where a forensic pathologist is presented with a tissue of unknown origin. Panels of such Th2-specific probes, can be used to identify tissue by species and/or by organ type.

In a similar fashion, these reagents, e.g., Th2-specific primers or probes can be used to screen tissue culture for contamination (i.e., screen for the presence of a mixture of different types of cells in a culture).

C. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. These applications are described in the subsections below.

1. Diagnostic Assays

One aspect of the present invention relates to diagnostic assays for detecting Th2-specific protein and/or nucleic acid expression as well as Th2-specific activity, in the context of a biological sample. An exemplary method for detecting the presence or absence of Th2-specific proteins in a biological sample involves obtaining a biological sample from a test subject and contacting the biological sample with a compound or an agent capable of detecting Th2-specific protein or nucleic acid (e.g., mRNA, genomic DNA) that encodes Th2-specific protein such that the presence of Th2-specific protein is detected in the biological sample. Results obtained with a biological sample from the test subject may be compared to results obtained with a biological sample from a control subject.

A preferred agent for detecting Th2-specific mRNA or genomic DNA is a labeled nucleic acid probe capable of hybridizing to Th2-specific mRNA or genomic DNA. The nucleic acid probe can be, for example, a full-length Th2-specific nucleic acid, such as the nucleic acid of SEQ ID NO: 1, 3, 5, 7, 9, 11, 13, or 15, or a portion thereof, such as a nucleic acid molecule of at least 15, 30, 50, 100, 250, or 500 nucleotides in length and sufficient to specifically hybridize under stringent conditions to Th2-specific mRNA or genomic DNA. Other suitable probes for use in the diagnostic assays of the invention are described herein.

A preferred agent for detecting Th2-specific protein is an antibody capable of binding to Th2-specific protein, preferably an antibody with a detectable label. Antibodies can be polyclonal, or more preferably, monoclonal. An intact antibody, or a fragment thereof (e.g., Fab or F(ab′)₂)can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect Th2-specific mRNA, protein, or genomic DNA in a biological sample in vitro as well as in vivo. For example, in vitro techniques for detection of Th2-specific mRNA include Northern hybridizations and in situ hybridizations. In vitro techniques for detection of Th2-specific protein include enzyme linked immunosorbent assays (ELISAs), Western blots, immunoprecipitations, and immunofluorescence. In vitro techniques for detection of Th2-specific genomic DNA include Southern hybridizations. Furthermore, in vivo techniques for detection of Th2-specific protein include introducing into a subject a labeled anti-Th2-specific antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

The invention also encompasses kits for detecting the presence of Th2-specific proteins in a biological sample (a test sample). Such kits can be used to determine if a subject is suffering from or is at increased risk of developing a disorder associated with aberrant expression of Th2-specific protein (e.g., an immunological disorder). For example, the kit can comprise a labeled compound or agent capable of detecting Th2-specific protein or mRNA in a biological sample and means for determining the amount of a Th2-specific protein in the sample (e.g., an anti-Th2-specific antibody or an oligonucleotide probe that binds to DNA encoding a Th2-specific protein, e.g., SEQ ID NO:1, 3, 5, 7, 9, 11, 13, or 15). Kits can also include instructions for observing that the tested subject is suffering from or is at risk of developing a disorder associated with aberrant expression of Th2-specific sequences if the amount of Th2-specific protein or mRNA is above or below a normal level.

For antibody-based kits, the kit can comprise, for example: (1) a first antibody (e.g., attached to a solid support) that binds to Th2-specific protein; and, optionally, (2) a second, different antibody that binds to Th2-specific protein or the first antibody and is conjugated to a detectable agent. For oligonucleotide-based kits, the kit can comprise, for example: (1) an oligonucleotide, e.g., a detectably labeled oligonucleotide, that hybridizes to a Th2-specific nucleic acid sequence or (2) a pair of primers useful for amplifying a Th2-specific nucleic acid molecule.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample contained. Each component of the kit is usually enclosed within an individual container, and all of the various containers are within a single package along with instructions for observing whether the tested subject is suffering from or is at risk of developing a disorder associated with aberrant expression of Th2-specific proteins.

2. Prognostic Assays

The methods described herein can furthermore be utilized as diagnostic or prognostic assays to identify subjects having or at risk of developing a disease or disorder associated with Th2-specific protein, Th2-specific nucleic acid expression, or Th2-specific activity. Prognostic assays can be used for prognostic or predictive purposes to thereby prophylactically treat an individual prior to the onset of a disorder characterized by or associated with Th2-specific protein, Th2-specific nucleic acid expression, or Th2-specific activity.

Thus, the present invention provides a method in which a test sample is obtained from a subject, and Th2-specific protein or nucleic acid (e.g., mRNA, genomic DNA) is detected, wherein the presence of Th2-specific protein or nucleic acid is diagnostic for a subject having or at risk of developing a disease or disorder associated with aberrant Th2-specific expression or activity. As used herein, a “test sample” refers to a biological sample obtained from a subject of interest. For example, a test sample can be a biological fluid (e.g., serum), cell sample, or tissue.

Furthermore, using the prognostic assays described herein, the present invention provides methods for determining whether a subject can be administered a specific agent (e.g., an agonist, antagonist, peptidomimetic, protein, peptide, nucleic acid, small molecule, or other drug candidate) or class of agents (e.g., agents of a type that decrease Th2-specific activity) to effectively treat a disease or disorder associated with aberrant Th2-specific expression or activity. In this manner, a test sample is obtained and Th2-specific protein or nucleic acid is detected. The presence of Th2-specific protein or nucleic acid is diagnostic for a subject that can be administered the agent to treat a disorder associated with aberrant Th2-specific expression or activity.

The methods of the invention can also be used to detect genetic lesions or mutations in a Th2-specific gene, thereby determining if a subject with the lesioned gene is at risk for a disorder characterized by aberrant cell proliferation and/or differentiation. In preferred embodiments, the methods include detecting, in a sample of cells from the subject, the presence or absence of a genetic lesion or mutation characterized by at least one of an alteration affecting the integrity of a gene encoding a Th2-specific-protein, or the misexpression of the Th2-specific gene. For example, such genetic lesions or mutations can be detected by ascertaining the existence of at least one of: (1) a deletion of one or more nucleotides from a Th2-specific gene; (2) an addition of one or more nucleotides to a Th2-specific gene; (3) a substitution of one or more nucleotides of a Th2-specific gene; (4) a chromosomal rearrangement of a Th2-specific gene; (5) an alteration in the level of a messenger RNA transcript of a Th2-specific gene; (6) an aberrant modification of a Th2-specific gene, such as of the methylation pattern of the genomic DNA; (7) the presence of a non-wild-type splicing pattern of a messenger RNA transcript of a Th2-specific gene; (8) a non-wild-type level of a Th2-specific-protein; (9) an allelic loss of a Th2-specific gene; and (10) an inappropriate post-translational modification of a Th2-specific-protein. As described herein, there are a large number of assay techniques known in the art that can be used for detecting lesions in a Th2-specific gene. Any cell type or tissue, preferably peripheral blood leukocytes, in which Th2-specific proteins are expressed may be utilized in the prognostic assays described herein.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the Th2-specific-gene (see, e.g., Abravaya et al. (1995) Nucleic Acids Res. 23:675-682). It is anticipated that PCR and/or LCR may be desirable to use as a preliminary amplification step in conjunction with any of the techniques used for detecting mutations described herein.

Alternative amplification methods include self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a Th2-specific gene from a sample cell can be identified by alterations in restriction enzyme cleavage patterns of isolated test sample and control DNA digested with one or more restriction endonucleases. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in a Th2-specific molecule can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). In yet another embodiment, any of a variety of sequencing reactions known in the art can be used to directly sequence the Th2-specific gene and detect mutations by comparing the sequence of the sample Th2-specific gene with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the Th2-specific gene include methods in which protection from cleavage agents is used to detect mismatched bases in RNA/RNA or RNA/DNA heteroduplexes (Myers et al. (1985) Science 230:1242). See, also Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more “DNA mismatch repair” enzymes that recognize mismatched base pairs in double-stranded DNA in defined systems for detecting and mapping point mutations in Th2-specific cDNAs obtained from samples of cells. See, e.g., Hsu et al. (1994) Carcinogenesis 15:1657-1662. According to an exemplary embodiment, a probe based on a Th2-specific sequence, e.g., a wild-type Th2-specific sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in Th2-specific genes. For example, single-strand conformation polymorphism (SSCP) may be used to detect differences in electrophoretic mobility between mutant and wild-type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). The sensitivity of the assay may be enhanced by using RNA (rather than DNA), in which the secondary structure is more sensitive to a change in sequence. In a preferred embodiment, the subject method utilizes heteroduplex analysis to separate double-stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324: 163); Saiki et al. (1989) Proc. Natl. Acad. Sci. USA 86:6230). Such allele-specific oligonucleotides are hybridized to PCR-amplified target DNA or a number of different mutations when the oligonucleotides are attached to the hybridizing membrane and hybridized with labeled target DNA.

Alternatively, allele-specific amplification technology, which depends on selective PCR amplification, may be used in conjunction with the instant invention. Oligonucleotides used as primers for specific amplification may carry the mutation of interest in the center of the molecule so that amplification depends on differential hybridization (Gibbs et al. (1989) Nucleic Acids Res. 17:2437-2448) or at the extreme 3′ end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g., in clinical settings to diagnose patients exhibiting symptoms or family history of a disease or illness involving a Th2-specific gene.

3. Pharmacogenomics

Agents, or modulators that have a stimulatory or inhibitory effect on Th2-specific activity (e.g., Th2-specific gene expression) as identified by a screening assay described herein, can be administered to individuals to treat (prophylactically or therapeutically) disorders associated with aberrant Th2-specific activity as well as to modulate the phenotype of an immune response. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of Th2-specific protein, expression of Th2-specific nucleic acid, or mutation content of Th2-specific genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (antimalarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes-CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of Th2-specific protein, expression of Th2-specific nucleic acid, or mutation content of Th2-specific genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a Th2-specific modulator, such as a modulator identified by one of the exemplary screening assays described herein.

4. Monitoring of Effects During Clinical Trials

Monitoring the influence of agents (e.g., drugs, compounds) on the expression or activity of Th2-specific genes (e.g., the ability to modulate aberrant cell proliferation and/or differentiation) can be applied not only in basic drug screening but also in clinical trials. For example, the effectiveness of an agent, as determined by a screening assay as described herein, to increase or decrease Th2-specific gene expression, protein levels, or protein activity, can be monitored in clinical trials of subjects exhibiting decreased or increased Th2-specific gene expression, protein levels, or protein activity. In such clinical trials, Th2-specific expression or activity and preferably that of other genes that have been implicated in for example, a cellular proliferation disorder, can be used as a marker of the immune responsiveness of a particular cell.

For example, and not by way of limitation, genes that are modulated in cells by treatment with an agent (e.g., compound, drug, or small molecule) that modulates Th2-specific activity (e.g., as identified in a screening assay described herein) can be identified. Thus, to study the effect of agents on cellular proliferation disorders, for example, in a clinical trial, cells can be isolated and RNA prepared and analyzed for the levels of expression of Th2-specific genes and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of Th2-specific genes or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (1) obtaining a preadministration sample from a subject prior to administration of the agent; (2) detecting the level of expression of a Th2-specific protein, mRNA, or genomic DNA in the preadministration sample; (3) obtaining one or more postadministration samples from the subject, (4) detecting the level of expression or activity of the Th2-specific protein, mRNA, or genomic DNA in the postadministration samples; (5) comparing the level of expression or activity of the Th2-specific protein, mRNA, or genomic DNA in the preadministration sample with the Th2-specific protein, mRNA, or genomic DNA in the postadministration sample or samples; and (vi) altering the administration of the agent to the subject accordingly to bring about the desired effect, i.e., for example, an increase or a decrease in the expression or activity of a Th2-specific protein.

C. Methods of Treatment

The present invention provides for both prophylactic and therapeutic methods of treating a subject at risk of (or susceptible to) a disorder or having a disorder associated with aberrant Th2-specific expression or activity. Additionally, the compositions of the invention find use in modulating the T-lymphocyte response. Thus, therapies for immune and respiratory disorders are encompassed herein.

1. Prophylactic Methods

In one aspect, the invention provides a method for preventing in a subject a disease or condition associated with an aberrant Th2-specific expression or activity by administering to the subject an agent that modulates Th2-specific expression or at least one Th2-specific gene activity. Subjects at risk for a disease that is caused, or contributed to, by aberrant Th2-specific expression or activity can be identified by, for example, any or a combination of diagnostic or prognostic assays as described herein. Administration of a prophylactic agent can occur prior to the manifestation of symptoms characteristic of the Th2-specific aberrancy, such that a disease or disorder is prevented or, alternatively, delayed in its progression. Depending on the type of Th2-specific aberrancy, for example, a Th2-specific agonist or Th2-specific antagonist agent can be used for treating the subject. The appropriate agent can be determined based on screening assays described herein.

2. Therapeutic Methods

Another aspect of the invention pertains to methods of modulating Th2-specific expression or activity for therapeutic purposes. The modulatory method of the invention involves contacting a cell with an agent that modulates one or more of the activities of Th2-specific protein activity associated with the cell. An agent that modulates Th2-specific protein activity can be an agent as described herein, such as a nucleic acid or a protein, a naturally-occurring cognate ligand of a Th2-specific protein, a peptide, a Th2-specific peptidomimetic, or other small molecule. In one embodiment, the agent stimulates one or more of the biological activities of Th2-specific protein. Examples of such stimulatory agents include active Th2-specific protein and a nucleic acid molecule encoding a Th2-specific protein that has been introduced into the cell. In another embodiment, the agent inhibits one or more of the biological activities of Th2-specific protein. Examples of such inhibitory agents include antisense Th2-specific nucleic acid molecules and anti-Th2-specific antibodies.

These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a Th2-specific protein or nucleic acid molecule. In one embodiment, the method involves administering an agent (e.g., an agent identified by a screening assay described herein), or a combination of agents, that modulates (e.g., upregulates or downregulates) Th2-specific expression or activity. In another embodiment, the method involves administering a Th2-specific protein or nucleic acid molecule as therapy to compensate for reduced or aberrant Th2-specific expression or activity.

Stimulation of Th2-specific activity is desirable in situations in which a Th2-specific protein is abnormally downregulated and/or in which increased Th2-specific activity is likely to have a beneficial effect. Conversely, inhibition of Th2-specific activity is desirable in situations in which Th2-specific activity is abnormally upregulated and/or in which decreased Th2-specific activity is likely to have a beneficial effect.

This invention is further illustrated by the following examples, which should not be construed as limiting.

### EXAMPLES

In vitro and in vivo studies of T cell activation have lead to the widely accepted hypothesis that in addition to signals delivered to the T cell receptor (TCR) by MHC-II/peptide complexes, costimulatory signals by antigen presenting cells (APCs) are critical for complete immune activation, the absence of which results in an abortive immune response (Schwartz (1990) Science 4961:1349-56). The most important costimulatory signal delivered to resting T cells occurs upon CD28 engagement by B7 molecules (Jenkins et al. (1991) J. Immunol. 8:2461-6; Harding et al. (1992) Nature 6370:607-9). In contrast to CD28, the second member of this family of molecules, CTLA-4 delivers a negative signal to the activated T cell, opposing CD28-mediated costimulation (Walunas et al. (1994) Immunity 1(5):405-413).

Recent in vitro experiments have suggested that the dependency on CD28/B7 mediated costiumulation is greatly influenced by the antigenic experience of the T cell. Thus, while naive CD4+ T cells require CD28-mediated signaling for IL-2 production and clonal expansion (Seder et al. (1994) J. Exp. Med. 1:299-304; McKnight et al. (1994) J. Immunol. 11:5220-5), optimal activation of recently activated T helper subsets occurs independent of CD28 ligation (Schweitzer and Sharpe (1998) J. Immunol. 6:2762-71). In addition, while CD28 stimulation has been reported to promote differentiation to the Th2 phenotype in vitro (Rulifson et al. (1997) J. Immunol. 15:658-65), studies performed in CD28 gene targeted mice have demonstrated that at least under some circumstances, normal Th2 effector immune responses can be generated, suggesting other costimulatory signals are important in Th2 effector function (Brown et al. (1996) J. Exp. Med. 3:803-10; Gause et al. (1997) J. Immunol. 158:4082-7; Wu et al. (1998) J. Exp. Med. 187:1151-1156).

In an attempt to identify novel candidate genes that may function as costimulators of T helper effector cells, subtractive libraries from activated murine Th1 vs. Th2 clones were generated. Using a subtractive hybridization-PCR approach on murine Th2 vs. Th1 mRNA, the murine orthologues for three novel human genes that are differentially expressed in the Th2 subset of T-helper cells were identified. The first of these orthologues, m1419, encodes a 3.6 Kb transcript (corresponding cDNA set forth in SEQ ID NO: 1) that is overexpressed in CD3/TCR-activated Th2 cells. The open reading frame (nt 126-1304) of this transcript encodes a predicted 392 amino acid, 42.8 kDa protein (SEQ ID NO: 2). A search of the nucleotide and protein databases revealed that the m1419 cDNA is novel. The only significant homology detected was to a Caenorhabditis elegans sequence (GenBank Accession Number AAB00590; Fulton and Gattung (1994) Nature 368:32-38; Waterston, R., Direct Submission, May 19, 1996, Genome Sequencing Center, Department of Genetics, Washington University, St. Louis, Mo., 63110 USA). The amino acid sequence for m1419 showed approximately 40% identity over 140 amino acids with the putative translation of one of these C. elegans sequences. A cDNA (corresponding to a 3.9 Kb transcript) for the corresponding human Th2-specific gene, h1419 (SEQ ID NO: 15), has been isolated; the open reading frame (nt 325-1479) of this cDNA encodes a 384 amino acid protein (SEQ ID NO: 16).

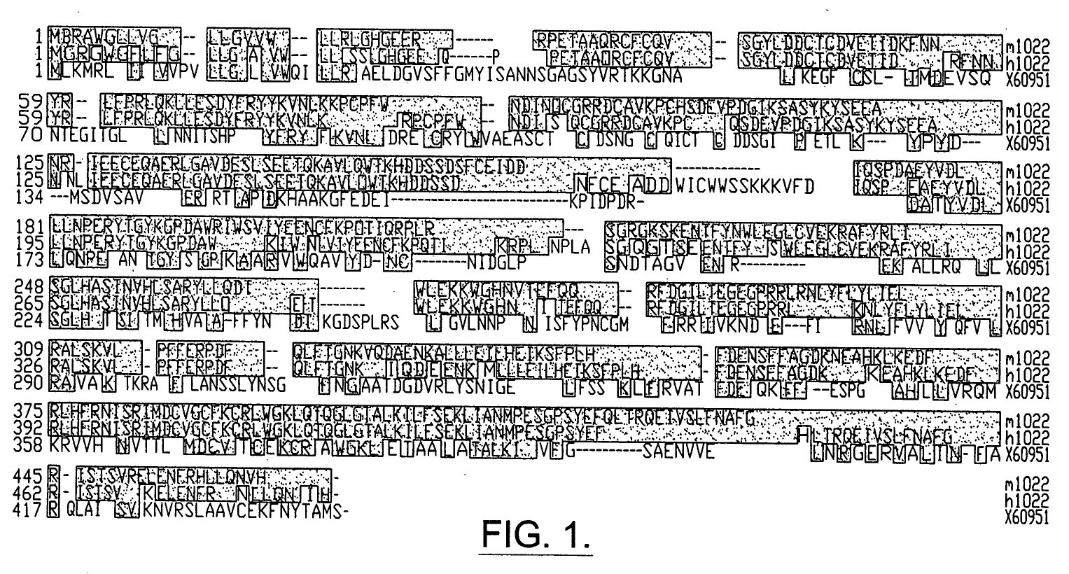

The second of these murine orthologues, m1022, encodes two transcripts, a shorter 1.6 Kb form (corresponding cDNA set forth in SEQ ID NO: 3) and a longer 4.4 Kb form (corresponding cDNA set forth in SEQ ID NO: 5), that are overexpressed in CD3/TCR-activated Th2 cells. These sequences differ only in their 3′-untranslated region, with the shorter form corresponding to nt 1-1587 of the longer form. The open reading frame (nt 36-1430) of both transcripts encodes a 464 amino acid, 54.2 kDa protein (SEQ ID NOs: 4 and 6) that is homologous (48.3% identity over a 60 amino acid overlap; see FIG. 1) with GenBank Accession Number CAA43286, which codes for the alpha core subunit of DNA polymerase from Trypanosoma brucei (Leegwater, Direct Submission, Jul. 19, 1991, International Laboratory for Research on Animal Disease, P.O. Box 30709, Nairobi, Kenya; Leegwater et al. (1991) Nucleic Acids Res. 19:6411-6447). A cDNA (corresponding to a 1.8 Kb transcript) for the corresponding human Th2-specific gene, h1022 (SEQ ID NO: 13), has been isolated; the open reading frame (nt 57-1502) of this cDNA encodes a 481 amino acid protein (SEQ ID NO: 14).

The third of these murine orthologues, m1228, encodes two transcripts, a shorter 2.1 Kb form (corresponding cDNA set forth in SEQ ID NO: 7) and a longer 3.3 Kb form (corresponding cDNA set forth in SEQ ID NO: 9), that are overexpressed in CD3/TCR-activated Th2 cells. These sequences differ only in their 3′-untranslated region, with the shorter form corresponding to nt 1-2080 of the longer form. The open reading frame (nt 40-642) of both transcripts encodes a predicted 200 amino acid, 22.7 kDa protein (SEQ ID NOs:8 and 10). A cDNA (corresponding to a 2.7 Kb transcript) for the corresponding human Th2-specific gene, h1228 (SEQ ID NO: 11), has been isolated; the open reading frame (nt 115-711) of this cDNA encodes a 198 amino acid protein (SEQ ID NO: 12). The h1228 protein and the corresponding m1228 protein are Ig superfamily members, which share 69% identity over their full-length amino acid sequences.

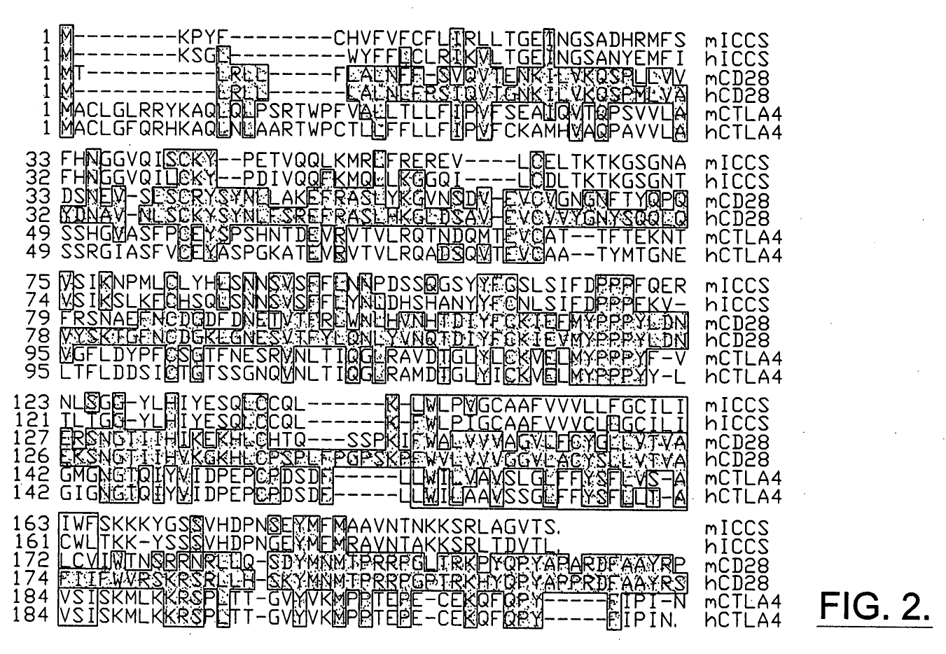

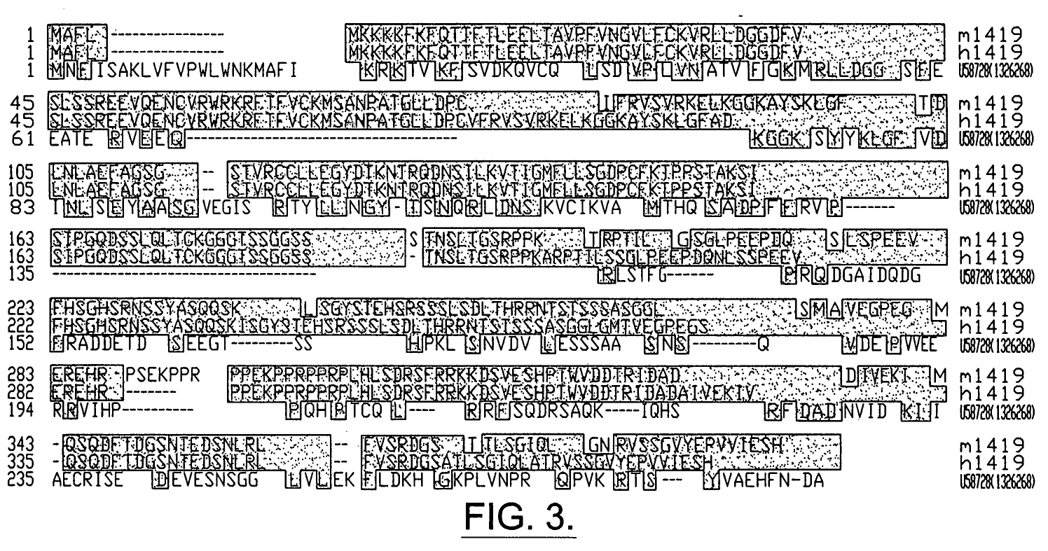

The predicted h1228 and m1228 proteins share homology to both human and murine CD28 and CTLA-4 (see FIG. 2). The h1228 sequence shares 33% identity with hCD28 and 26% identity with hCTLA-4. The murine orthologue m1228 shares 36.5% identity with mCD28 and 38.5% identity with mCTLA-4. The human homologue of m1228 has recently been designated ICOS, the third member of the CD28/CTLA-4 family (Hutloff et al (1999) Nature 6716:263). The m1228 gene and protein are thus also referred to as mICOS in Example 6 below. Examination of the amino acid sequence of m1228 and of h1228 revealed 4 conserved cysteine residues (amino acid residues 42, 63, 83, and 137 of SEQ ID NOs: 8 and 10; amino acid residues 41, 62, 82, and 135 of SEQ ID NO: 12). There is a conserved PPP motif common in CD28 and its related homologue CTLA-4. This motif, which is in the extracellular domain, is found in human CD28 as MYPPPY (amino acid residues 117-122 of SEQ ID NO: 19), in human CTLA-4 as MYPPPY (amino acid residues 134-139 of SEQ ID NO: 21), in m1228 as FDPPPF (amino acid residues 114-119 of SEQ ID Nos:8 and 10), and in h1228 as FDPPPF (amino acid residues 113-118 of SEQ ID NO: 12). In addition, the 1228 sequence contains a YXXM motif (where X can be any amino acid) common in CD28 and CTLA-4 that appears to be required for CD28-mediated phosphatidylinoisitol 3-kinase (PI-3K) activity. This motif, which is in the intracellular domain of CD28 and 1228, is involved in the signaling pathways. This sequence is found in human CD28 as YMNM (amino acid residues 191-194 of SEQ ID NO: 19), in human CTLA-4 as YVKM (amino acid residues 201-204 of SEQ ID NO: 21), in m1228 as YMFM (amino acid residues 181-184 of SEQ ID NOs: 8 and 10), and in h1228 as YMFM (amino acid residues 178-181 of SEQ ID NO: 12). Taken together, these data suggest that h1228 and the corresponding m1228 function as novel Th2-specific costimulatory molecules.

The methods employed to identify the murine orthologues and the corresponding human genes and to characterize their expression patterns are described in the following examples.

### Example 1

### Th1 and Th2 mRNA Generation

Mice expressing the transgene for the DO 11.10 αβ-TCR, which recognizes residues 323-339 of chicken ovalbumin (OVA) in association with I-A^{d}, were maintained on the BALB/c background. The mice were housed in an animal facility in microisolator cages under pathogen-free conditions in accordance with institutional and state guidelines.

DO 11.10 TCR-transgenic CD4⁺ T cells were cultured in complete RPMI 1640 with OVA 323-339 (1 mM) and mitomycin C-treated BALB/c spleen cells. For Th1 phenotype development, 10 ng/ml of recombinant murine IL-12 (R & D Systems) and neutralizing anti-IL-4 mAb (11B11) (10 μg/ml) were added, and for Th2 development, recombinant murine IL-4 (R & D Systems) and neutralizing polyclonal anti-murine IL-12 (10 μg/ml) (TOSH-2, Endogen, Cambridge, Mass.) were added and stimulated with the antigenic peptide OVA 323-339. Cells were cultured for three rounds of antigenic stimulations under polarizing conditions. Cells were then washed and cultured in mIL-2 (10 ng/ml) for 48 hours. Dead cells were then removed by gradient centrifugation and cells (5×10⁶/well) plated on immobilized CD3 (2C11, 10 μg/ml) on 6 well plates in the presence of hIL-2 for 6, 24, or 48 hours. Resting cells were cultured in IL-2 for 48 hours, but not stimulated, and were used as nonactivated control cells.

### Example 2

### Subtractive Hybridization Protocol

Poly-A+ RNA was isolated using the FastTrack mRNA isolation kit (Invitrogen, San Diego Calif.). A Th2-specific library was generated using the Clontech PCR-Select cDNA Subtraction Kit (Clontech, Palo Alto, Calif.) according to the attached protocol. 2.0 μg of poly-A+ RNA derived from activated Th2 cells was used as “tester”, and 2.0 μg of poly-A+ RNA derived from activated Th1 cells was used as “driver”. PCR products from the resulting library were cloned into the PCRII cloning vector (Invitrogen, San Diego, Calif.). Plasmid DNA from individual clones was spotted onto nylon filters, and DNA was denatured by soaking the filters sequentially in 1.5M NaCl, 0.5M NaOH followed by 1.5M NaCl, 0.5M Tris pH 8.0. Single stranded probes were prepared from Poly-A+ RNA from the two cell populations, by reverse transcription using ³²P-labeled dCTP. Clones showing a differential hybridization to Th2 vs. Th1 cDNA were selected and sequenced. Upon further analysis, the cDNA clones termed m1419, m1022, and m1228 were identified.

### Example 3

### mRNA Expression of cDNA Clones m1419, m1022, and m1228

Differential expression of each of the three murine cDNA clones obtained from this library was confirmed by Northern Blot analysis using standard techniques (Maniatis et al. (1982) Molecular Cloning: A Laboratory Manual (Cold Spring Harbor Laboratory Press, Cold Spring Harbor, N.Y.)), with 0.1-1.0 μg of poly-A+ RNA, or 5.0 μg of total RNA, purified from two independent preparations of Th1 and Th2 cells polarized as described above. For each clone, cDNA probes were labeled with ³²P-dCTP using standard techniques (Maniatis et al., supra). The probe for clone m1419 (SEQ ID NO: 1) was of a 713 bp cDNA consisting of the region from nt 2,310 to nt 3,000 of the full-length m1419 cDNA. The probe for clone m1022 (SEQ ID NOs: 3 and 5) was a 482 bp cDNA consisting of the region from nt 470 to nt 970 of the full-length m1022 cDNA. The probe for clone m1228 (SEQ ID NOs: 7 and 9) was a 965 bp cDNA consisting of the region from nt 87 to nt 1051 of the full-length m1228 cDNA.

Expression of mRNA for each of these three clones was determined in (a) Th1 and Th2 mRNA 48 hours after CD3/TCR crosslinking, or (b) resting cells and 6 hours post CD3 activation in Th1 and Th2 populations. Although resting Th1 and Th2 cells both expressed the m1419 mRNA, this transcript was clearly up-regulated in the Th2 population, and down-regulated in the Th1 population.

Expression of mRNA for each of these three clones mRNA was also analyzed in normal mouse tissues obtained from untreated or LPS-treated animals. Northern Blot analysis was performed using 0.5 μg of poly-A+ RNA derived from the following tissues: liver, lung, brian, kidney, heart, skeletal muscle, skin, bone, lymph node, spleen and thymus. The m1419 mRNA was detected in only the activated lymph node, and to a lesser extent, in brain. Expression of clone m1022 was not detected in either normal mouse tissues or in tissue obtained from LPS treated animals.

### Example 4

### Full-Length cDNA Cloning

A cDNA library was prepared from murine Th2 cells derived from the same poly-A+ RNA used for the subtracted Th2 library described above. cDNAs were cloned into a lambda phage expression vector, γZIPLOX (Gibco BRL, Gaithersburg, Md.) using the SaII and NotI cloning sites. The cDNAs corresponding to the transcripts identified with the respective m1419, m1022, and m1228 probes were isolated from this library.

### Example 5

### Isolation of h1228, 1022, and 1419

The human 1228 (cDNA set forth in SEQ ID NO: 11), human 1022 (cDNA set forth in SEQ ID NO: 13), and human 1419 (cDNA set forth in SEQ ID NO: 15) clones were obtained by screening a human mixed lymphocyte library with the corresponding murine probes. The materials and methods for making the library were identical to the methods used for obtaining the mouse clone, except for the source of the RNA. The library was screened with a probe that comprised the entire coding region of the murine gene for either 1228 (SEQ ID NO: 9, nt 40-642), 1022 (SEQ ID NO: 5, nt 36-1430), or 1419 (SEQ ID NO: 1, nt 126-1304). Hybridization was performed at 40° C. (instead of 65° C.), and the filters were washed under lower stringency (0.5×SSC, 0.1% SDS at room temperature).

### Example 6

### Further Characterization of the m1228 Clone

The murine clone identified as m1228, referred to below as mICOS, was selected for further characterization of its role in Th2 immune responses.

### Materials and Methods

Expression of mICOS

The restricted pattern of mICOS expression was extended using real time quantitative PCR analysis (Taqman™). In brief, an oligonucleotide probe was designed to anneal to the mICOS gene between two PCR primers. The probe was then fluorescently labeled with FAM (reporter dye) on the 5′ end and TAMRA (quencher dye) on the 3′ end. A similar probe and PCR primers were designed for mGAPDH. The probe for this gene incorporated VIC as the reporter dye. PCR reactions were run that included the primers and probes for these two genes, as well as cDNA made from various cells and tissues. As the polymerase moves across the gene during the reaction, it cleaves the dye from one end of each probe, which causes a fluorescent emission that is measured by the Sequence Detector 7700. The emissions recorded for each cDNA can then be converted into the level of expression for mICOS normalized to the expression of mGAPDH. Splenic CD4, CD8, and CD3 cells were purified by negative selection (R & D Systems). B cells were isolated by positive selection by labeling cells with rat anti-CD19 and separating the cell using rat IG coupled beads (MACS Microbeads Miltenyi Biotec, CA). Neutrophils were obtained from the peritoneum after thioglycollate treatment, and macrophages were obtained as the adherent fraction of peritoneal cells. Finally, eosinophils (92% purity) were isolated as the nonadherent population of peritoneal cells obtained from IL-5 transgenic mice.

Mapping mICOS to Chromosome 1

mICOS-specific primers (Forward-AACCTTCTAGTCCTTTGGTCTGC, SEQ ID NO:23; Reverse-GGCCCAGGCTACAGGCTG, SEQ ID NO: 24) were used to amplify a 159 bp PCR product from both C57BL/6J and the wild-derived Mus spretus strain SPRET/EiJ. Single stranded conformation polymorphism (SSCP) analysis identified a polymorphism between C57BL/6J and M. spretus. The genetic segregation of the M. spretus allele was followed in 181 progeny of a (C57BL/6J×M. spretus)×C57BL/6 mapping panel by SSCP. The segregation pattern of the M. spretus allele was compared with the segregation pattern of 359 other genetic loci that have been mapped in this backcross panel. mICOS mapped to murine chromosome 1, approximately 16.2±2.75 cM distal to the microsattelite marker D1MIT4 and 15.91±2.76 cM proximal of the marker D1MIT8. Further, using SSCP markers designed from the published sequence of the CD28 and CTLA-4 genes, mICOS was found to completely cosegregate with these genes on this mapping panel (no recombinations detectable among all three genes).

Generation of mICOS-Ig Fusion Proteins

A DNA sequence containing the extracellular domain of mICOS was PCR-amplified and cloned into a vector containing the CD5 signal sequence and the human IgG1 constant region (mICOS-Ig). COS cells were transiently transfected using lipofectamine™ (GIBCO) and the recombinant protein purified over a protein A column. The purity of ICOS-Ig was subsequently assessed by coomassie-stained SDS-PAGE and was determined to be greater than 90%. The identity of the ICOS-Ig was further confirmed by mass spectrometry by comparing the trypsin peptides generated from the extracted gel band to a theoretical trypsin digest (peptide mass fingerprinting by MALDI-TOF analysis).

Binding of ICOS-Ig to Transfectants and Antigen Presenting Cells (APCs)

EL-4 cell lines expressing murine B7-1 or B7-2 were incubated with 100 μg/ml of ICOS-Ig, 10 μg/ml CD28-Ig or 1 μg/ml of CTLA-4-Ig (R & D Systems) for 30 minutes at 4° C. after preincubation with FcBlock™ (Pharmingen). Human Ig was used as a negative control for binding. Cells were then incubated with rat anti-human Ig-FITC and fluorescence determined by flow cytometry (FACstar Vantage, Becton Dickinson,) by gating on viable cells. In addition, we investigated binding of ICOS-Ig to bone marrow-derived dendritic cells, prepared as described in detail elsewhere (Inaba et al. (1992) J. Exp. Med. 6:1693-1702) or on purified splenic B cells. Binding was determined on resting cells or 24 hours after activation with soluble anti-CD40 mAb (10 μg/ml).

Generation of Stable Jurkat Cells Expressing mICOS

Jurkat cells were transfected by electroporation with 2 μg mICOS-Flag DNA subcloned in pcDNA3 (Invitrogen). After a 2-week drug selection, high Flag-positive expressing cells were sorted by flow cytometry (FACStar Vantage, Beckon Dickinson), subdloned, and stable lines generated. mICOS and empty vector cells were then selected on the basis of comparable surface expression of hCD3 and hCD28.

Immunoprecipitation and Immunoblotting

For immunoprecipitations, Jurkat mICOS-Flag and empty vector transfected cells were either left unstimulated or stimulated with anti-CD28 (10 μg/ml, 4B10) or anti-Flag mAb (10 μg/ml) and rabbit anti-mouse antibody (5 μg/ml) for 2 and 7.5 minutes. Cells were then lysed in ice cold lysis buffer containing 1% TritonX-100 (v/v) in 20 mM Tris-HCl pH 8.3, 150 mM NaCl. The lysis buffer contained 1 mM PMSF, 1 mM Na₄VO₃, 10 mM NaF and 1 mM Na₄P₂O₇. Lysates were incubated for 20 minutes on ice before centrifugation at 1500×g for 15 minutes at 4° C. Postnuclear lysates were incubated for 1 hour with agitation at 4° C. with the indicated monoclonal antibody. ProteinA-Sepharose beads (30 μt, Pharmacia), swollen and washed in lysis buffer were added and incubated for 1 hour at 4° C. The beads were washed three times in cold lysis buffer, and proteins were eluted by boiling for 5 minutes in SDS sample buffer, separated by SDS-PAGE, and transferred to nitrocellulose for immunoblotting. The membranes were blocked with 5% milk in TBS (10 mM Tris-HCl, pH 7.6, 150 mM NaCl) and incubated with p85 antiserum. Bound antibody was revealed with horseradish peroxidase (HRP)-conjugated anti-rabbit antibody using enhanced chemilluminescence (ECL, Amersham). Results are summarized below.

Lipid Kinase Assay

Jurkat mICOS-Flag and empty vector transfected cells were either left unstimulated or stimulated with anti-CD28 (10 μg/ml, 4B10) or anti-Flag mAb (10 μg/ml) and rabbit anti-mouse antibody (5 μg/ml) for 2 and 7.5 minutes. Cells were solubilized in 1% Triton X-100 (v/v)-based lysis buffer with protease and phosphatase inhibitors and subjected to precipitation as described above. Immune complexes were washed three times with the lysis buffer, three times with 100 mM Tris-HCl, pH 7.5 with 0.5 m LiCl, and twice with TNE (10 mM Tris-HCl, pH 7.5, 150 mM NaCl, and 1 mM EGTA). The lipid kinase reaction was carried out on the beads using soybean PI liposomes and γ[³²] ATP [20 μCi) as previously described (Prasad et al. (1994) Proc. Natl. Acad. Sci. USA 91:2834-2838).

Inhibition of T Effector Function by mICOS-IG

To determine the effect of mICOS in activation of effector cells, CD4 cells were differentiated to Th1 and Th2 cells and then reactivated in 96 well plates (5×10⁴/well) together with mitomycin C-treated 1×10⁵ splenocytes/well and stimulated with 10 μg/ml OVA peptide in the presence of either hIg or mICOS-IG (1-100 μg/ml). IL-4, IL-5 and IFN-γ were measured by specific ELISA.

Secondary T Cell Dependent B Cell Immune Responses

Male Balb/c mice (15-20 g) were immunized in the footpad on day 0 with 10 μg OVA in 4 mg alum. On day 8, mice were boosted with 100 μg OVA/alum administered subcutaneously. ICOS-Ig or human IgG (100 μg/mouse) was i.p. administered on day 7, 8, and 9. On day 18, mice were bled, and antigen specific IgE and IgG1 were measured by specific ELISA (Pharmingen).

Active Immunization Protocol for Aeroallergen Challenge

Male Balb/c mice (15-20 g) were immunized intraperitoneally with 10 μg of OVA in 4 mg alum (Serva, Heidelburg, Germany) on day 0 and day 14. On day 21, mice were challenged with aerosolized OVA (50 mg/ml) for 20 minutes. Control mice were challenged with PBS instead of OVA. One hour prior to antigen sensitization and challenge, mice were injected intranasally with 100 μg of CTLA-4-Ig, ICOS-Ig, or human Ig. Forty-eight hours later, the tracheas were cannulated and a bronchoalveolar lavage (BAL) performed. Cytospin preparations were prepared, stained with Giemsa reagent, and a total of 200 cells counted differentially using standard morphological criteria.

Adoptive Transfer of Antigen Specific T Cells In Vivo

Recipient normal Balb/C mice were injected i.v. with 2×10⁶ Th1 or Th2 effector cells. Twenty-four hours later, mice were exposed to an aerosol of ovalbumin (50 mg/ml) for 20 minutes on two consecutive days. One hour prior to allergen exposure, recipient mice were injected i.v. with either 100 μg of mICOS-Ig, CTLA-4-Ig, or hIg (Sigma, St. Louis, Mo.). Twenty four hours later, a BAL was performed and cytokine levels in the lavage fluid measured by ELISA. Lungs were removed 24 hours after antigen challenge, inflated with 10% neutral buffered formalin, and paraffin embedded. Four-micron sections were prehybridized for 2-4 hours, after which ³⁵S-labeled mICOS sense and antisense riboprobes (1.2×106 cpm/slide) were added and hybridized overnight. Slides were dipped in emulsion, exposed for 2 weeks, developed, and counterstained with haematoxylin.

Measurement of Airway Hyperresponsiveness

Airway responsiveness was measured in both Th1 and Th2 recipient mice 24 hours after the last aerosol challenge by recording respiratory pressure curves by whole body plethysmography (Buxco, EMKA Technologies, France) in response to 20 mg/ml of methacholine inhaled for 1 minute. Airway responsiveness was expressed in enhanced pause (Penh), a calculated value, which correlates with measurement of airway resistance, impedance, and intrapleural pressure in the same mouse. Penh=(Te/TR1)×Pef/Pif (Te=expiration time, Tr=relaxation time, Pef=peak expiratory flow, Pif=peak inspiratory flow), as described in detail elsewhere (Tsuyuki et al. (1977) J. Exp. Med. 9:1671-1679; Hamelmann et al. (1977) Am. J. Respir. Crit. Care Med. 156:766-775).

### Results and Discussion

Expression of mICOS

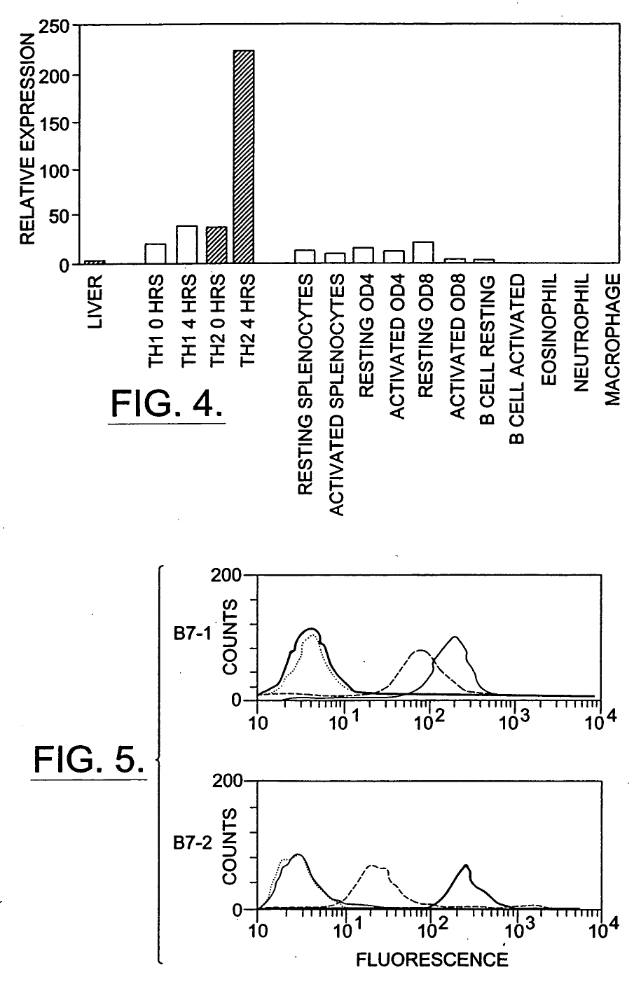

Murine ICOS (mICOS) exhibits 69% homology to the human gene (FIG. 2). Unlike CD28, which is expressed in comparable levels on T helper subsets, northern blot analysis confirmed the nylon microarray specific hybridization data and demonstrated that mICOS was constitutively expressed in Th2, but not Th1 clones. Similarly, mICOS was overexpressed upon CD3/TCR crosslinking in Th2 cells derived from common T helper precursor (Thp) cells as compared to activated Th1 cells. mICOS was not expressed by B cells, resting CD4, CD8, macrophages, neutrophils, or eosinophils as determined using real time quantitative PCR analysis (Taqman™, Perkin Elmer) (FIG. 4). Using single stranded conformation polymorphism analysis (SSCP), mICOS mapped to chromosome 1, cosegregating with CD28 and CTLA-4, further supporting the claim that ICOS represents the third member of the CD28 family.

ICOS Binds a Non-B7 Counterreceptor on Activated B Cells and Dendritic Cells

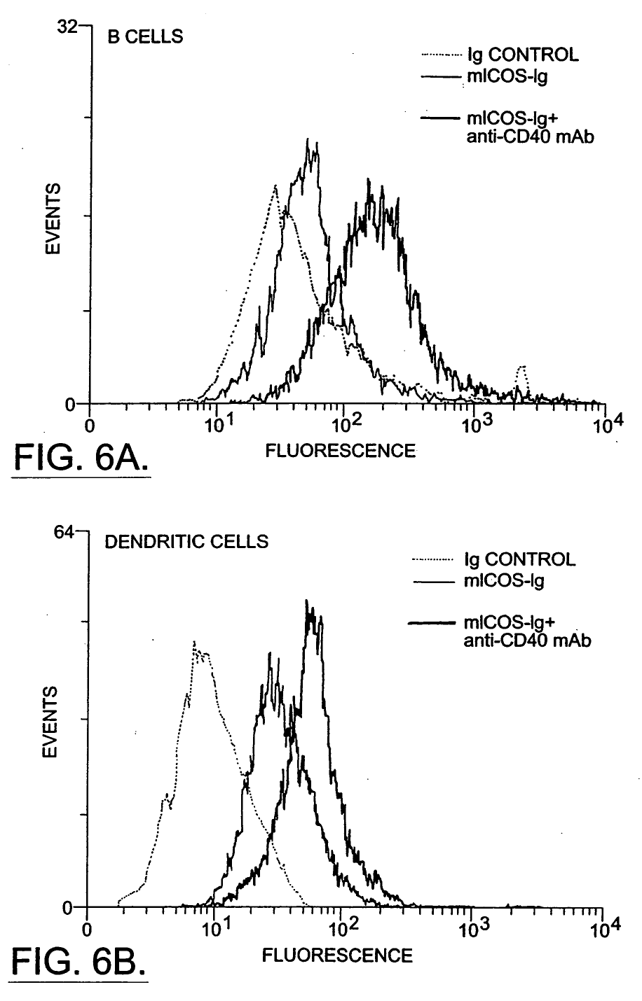

mICOS, like its human counterpart, has an FDPPPF motif at positions 114-119, replacing the putative B7 MYPPPY binding domain found in both CD28 and CTLA-4. However despite the homology between CD28 and mICOS, a soluble protein consisting of the extracellular portion of mICOS and human IgG1 failed to exhibit any binding to either mB7-1 or mB7-2 stable transfectants (FIG. 5). These observations support previous reports that mutation of the tyrosine residues of the putative B7 MYPPPY binding domain of CD28 to phenylalanine at corresponding positions 115 and 119 of mICOS results in a complete loss of B7 binding (Kariv et al. (1996) J. Immunol. 1:29-38). However, a putative binding site for mICOS-Ig was identified on CD40-stimulated, but not resting B cells (FIG. 6A), and on bone marrow-derived dendritic cells (FIG. 6B), but not resting or activated CD3+ T cells.

ICOS and CD28 Share Some Common Signaling Mechanisms

In a further comparison of mICOS and CD28, the receptors were compared for their ability to interact with intracellular signaling proteins. CD28 has previously been shown to bind the lipid kinase phosphatidylinositol 3-kinase (PI-3K) by means of a phosphotyrosine based motif, pYMNM, which serves as a binding domain for the Src homology domain (SH2 domain) of PI-3K (Truitt et al. (1994) J. Exp. Med. 3:1071-1076; Prasad et al. (1994) Proc. Natl. Acad. Sci. USA 91:2834-2838). Upon binding to CD28, PI-3K is then sequestered to the inner face of the plasma membrane, where it can act on its target substrates. mICOS possesses a similar YMFM motif (residues 181-184) and is therefore also potentially suitable for binding to the p85 subunit of PI-3K. Immunoprecipitation of mICOS, like CD28, co-precipitated a 85 kDa band that corresponds to the p85 subunit of PI-3K as detected by anti-p85 immunoblotting, and like CD28, increased lipid kinase activity upon crosslinking the mICOS-Flag epitope. Deletion of the cytoplasmic tail (mICOS d180C) in mICOS transfectants ablated both PI-3K association and lipid kinase activity. These data demonstrate that CD28 and mICOS share some common downstream signaling elements. However, while CD28 pYMNM site also binds the SH2 domain of Grb-2, albeit at 10-fold lower activity than PI-3K, mICOS failed to recruit Grb-2, supporting previous data that the arginine residue at position 191 is required for Grb-2 association (Schneider et al. (1995) Eur. J. Immunol. 4:1044-1050). Nevertheless, despite some common signaling mechanisms, mICOS-Ig, unlike CTLA-4-Ig, failed to inhibit either antigen-specific proliferation and had only a minor effect on IL-2 secretion from antigen-stimulated naive CD4+ T cells, suggesting that mICOS plays a role distinct from CD28 in T cell activation (Table 1).

Table 1. Signaling through CD28, but not ICOS, is required for antigen specific proliferation and IL-2 production. CD4+ antigen specific cells were stimulated with OVA peptide (10 μg/ml) in the presence of either hIgG, CLTA-4-Ig, or mICOS-Ig (100 μg/ml). Proliferation was measured on d1, d2, d3, and d4 after stimulation using ³H-thymidine incorporation. IL-2 levels were measured in the supernatants on d3 by specific ELISA. Significance was determined by a Student's T-test; a value of p<0.05 is considered significant and is indicated by *.

| Proliferation (cpm) | | | | | IL-2 (ng/ml) |
|---|---|---|---|---|---|
| | d1 | d2 | d3 | d4 | d3 |
| hIgG | 228 | 13497 | 20367 | 2066 | 45 ± 2 |
| mICOS-Ig | 274 | 15913 | 21766 | 3432 | 32 ± 1.4* |
| CLTA-4-Ig | 183 | 8635* | 5637* | 1619 | 4.0 ± 0.1* |

Inhibition of ICOS Attenuates Th2 Cytokine Production In Vitro

In vitro studies using CTLA-4-Ig have suggested that CD28/B7 costimulation is required not only for IL-2 production, but also for priming naive CD4+ T cells for IFN-γ and IL-4 production (Seder et al. (1994) J. Exp. Med. 1:299-304; McKnight et al. (1994) J. Immunol. 11:5220-5225). However, signals delivered by CD28 are not required for optimal secretion of either IFN-γ or IL-4 secretion from recently activated T helper effector subsets (Schweitzer and Sharpe (1998) J. Immunol. 6:2762-2771). Given the overexpression of mICOS in Th2 effector cells, it was hypothesized that mICOS may provide a CD28 independent costimulatory signal specific for Th2, but not Th1 effector cells.

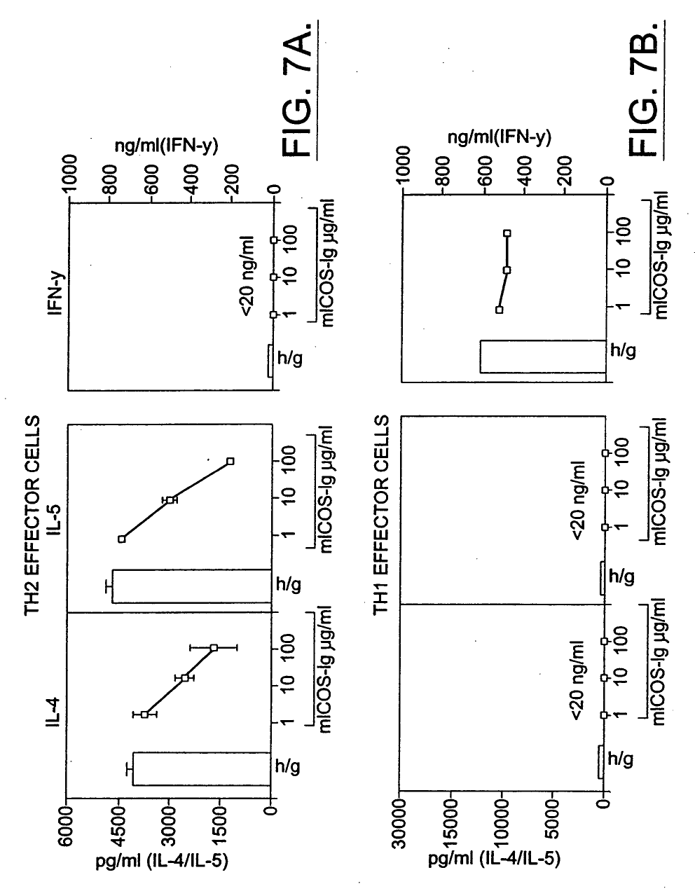

To address this issue, Th1 and Th2 effector cells were generated from common antigen-specific Thp cells under the influence of IL-12+anti-IL-4 mAbs and IL-4+anti-IL-12 mAb, respectively. To determine whether mICOS plays a critical role as a signaling molecule, experiments were performed using mICOS-Ig fusion protein that directly competes with membrane bound mICOS for binding with its putative ligand and hence blocks mICOS signaling. mICOS-Ig inhibited the production of IL-4 and IL-5 from Th2 cells (FIG. 7A) in a dose dependent manner, but failed to modif IFN-γ secretion from Th1 effector cells (FIG. 7B), when compared to hig-treated cells (closed bars). These data suggest that mICOS effectively replaces the CD28 signal and can provide a costimulatory signal specific for cytokine production from Th2 effector cells. Taken together, the data support previous observations generated in CD28 gene-targeted mice that fail to support the existence of alternative ligands for CD28 (Green et al. (1994) Immunity 6:501-508) and at the same time reconcile the limited role of CD28/B7 axis in Th2 immune deviation (Brown et al. (1996) J. Exp. Med. 3:803-810).

Regulation of T Cell Dependent B Cell Antibody Production by ICOS

While CTLA-4-Ig has been reported to be effective in inhibiting a number of immune responses in vivo when administered at the time of initial T cell activation, delaying CTLA-4-Ig treatment has in some situations, been reported to be ineffective (Corry et al. (1997) J. Immunol. 9:4142-4148; Sayegh et al. (1995) J. Exp. Med. 5:1869-1874). Likewise, although CTLA-4-Ig is effective in inhibiting a primary immune responses (Lu et al. (1995) J. Immunol. 154:1078-1087; Harris et al. (1999) Eur. J. Immunol. 29(1):311-316), some studies have shown that secondary immune responses cannot be fully suppressed by administration of CTLA-4-Ig (Tang et al. (1996) J. Immunol. 1:117-125; Gause et al. (1996) J. Immunol. 158:4082-7; Harris et al. (1999) Eur. J. Immunol. 29(1):311-316), consistent with B7 independent activation of effector cells.

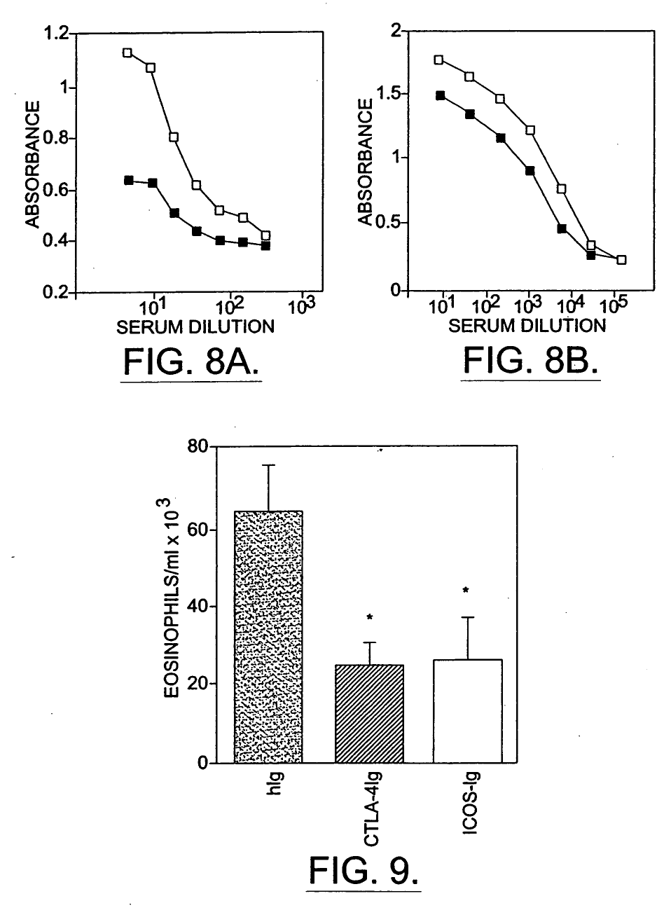

To address whether ICOS can regulate effector responses, a series of in vivo experiments using ICOS-Ig fusion protein were performed. Initially the contribution of ICOS to T cell-dependent B cell antibody production in a secondary immune response was studied by measuring antigen-specific antibodies after soluble antigen/adjuvant immunization. Administration of ICOS-Ig resulted in a marked attenuation of antigen-specific IgE and modestly reduced IgG1 levels by approximately 10-fold (FIG. 8). As the production of IgE/IgG1 is regulated by IL-4 (Snapper et al. (1988) J. Exp. Med. 167:183-196; Finkelman et al. (1990) Annu. Rev. Immunol. 8:303-333; Kopf et al. (1993) Nature 18:362, 245-248), these data provide the first in vivo evidence that ICOS can regulate T cell-dependent humoral immune responses.

Attenuation of Lung Mucosal Inflammation by ICOS-Ig

Allergen provocation of sensitized mice results in an Th2-dependent lung inflammation response. It has previously been reported that CTLA-4Ig can inhibit lung mucosal immune responses that are characterized by eosinophilic inflammation of the airways following aeroallergen provocation (Tsuyuki et al. (1997) J. Exp. Med. 9:1671-1679) and others (Padrid et al. (1998) Am. J. Respir. Cell. Mol. Biol. 4:453-462; Keane-Myers et al. (1997) J. Immunol. 158:2042-2049). To directly compare the roles of ICOS and CD28, either ICOS-Ig or CTLA-4-Ig was administered directly into the lungs, 1 hour prior to allergen challenge. The degree of lung inflammation was then measured by analysis of the cellular composition of the bronchoalveolar lavage fluid. Under these circumstances, ICOS-Ig was at least as effective as CTLA-4-Ig in suppressing airway inflammation, reducing the number of eosinophils by greater than 70% (FIG. 9).

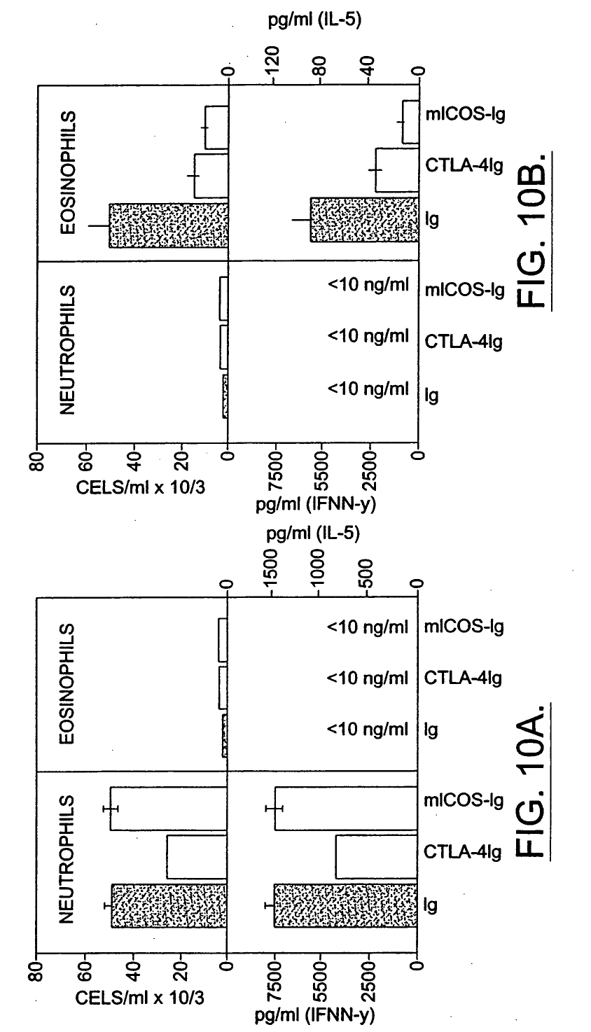

Due to the complexity of this active model of mucosal inflammation and the number of different pathways that have been implicated (for reviews, see Anderson and Coyle (1994) Trends Pharmacol. Sci. 15:324-332; Wills-Karp (1999) Annu. Rev. Immunol. 17:255-281), the contribution of mICOS to an in vivo lung mucosal immune response mediated exclusively by activation of antigen-specific cells generated in vitro was addressed in a Th1 and Th2 cell adoptive transfer model of lung mucosal immunity (Chon et al. (1997) J. Exp. Med. 10: 1737-1747). Aeroallergen provocation of mice transfused with antigen-specific Th1 or Th2 cells leads to the secretion of either IFN-γ or IL-5 and IL-4 and is associated with either a neutrophilic or eosinophilic lung mucosal inflammatory response, respectively (Chon et al. (1997) J. Exp. Med. 10: 1737-1747). In situ hybridization with an mICOS antisense riboprobe revealed a marked induction of mICOS and mRNA in the lungs after antigen challenge of Th2, but not Th1 recipient mice. Systemic administration of mICOS-Ig in OVA-exposed Th2 recipient mice inhibited the production of IL-5 and markedly suppressed eosinophilic inflammation of the airways by greater than 80% (FIG. 10B). In marked contrast, inhibition of mICOS-Ig did not modify Th1 effector responses as revealed either by IFN-γ secretion or Th1-mediated neutrophilic lung inflammation (FIG. 10A). These data suggest that inhibiting ICOS selectively abrogates the pathological consequences of activation of antigen-specific Th2, but not Th1 effector cells during an allergic lung mucosal inflammatory response. In contrast, administration of CTLA-4-Ig inhibited both Th1- and Th2-mediated inflammation and cytokine production, the latter being more sensitive to suppression than Th1-mediated inflammation.

ICOS is Critical for Th2-, but Not Th1-Mediated Altered Airway Responsiveness

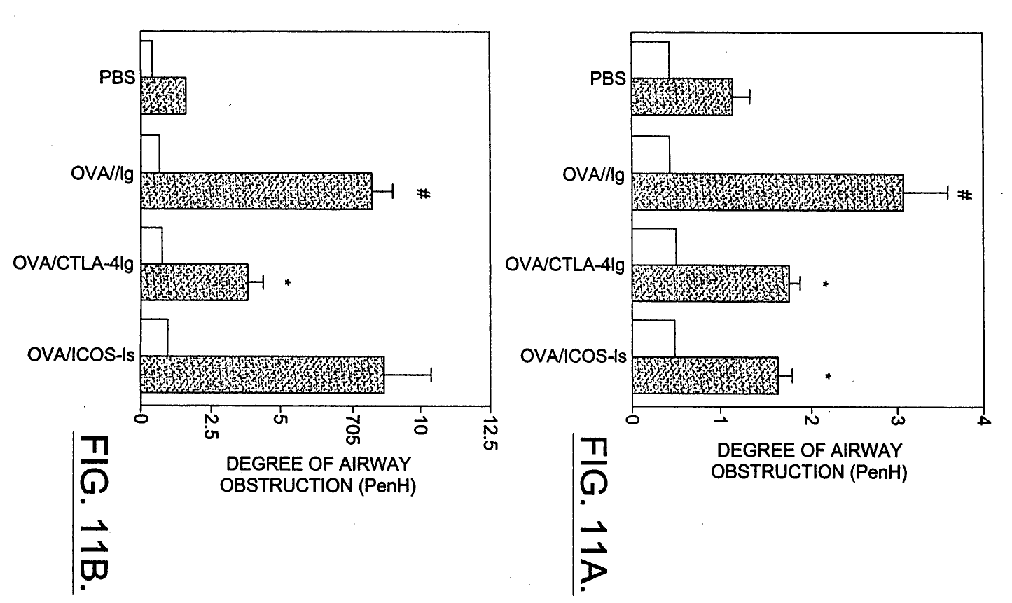

Airway hyperresponsiveness to non-specific stimuli such as methacholine is a characteristic feature of bronchial asthma. While prolonged (7-10 days) aeroallergen results in airway hyperresponsiveness only in Th2-recipient mice (Cohn et al. (1997) J. Ex└. Med. 10: 1737-1747), acute aerochallenge (2 days) results in a dramatic Th1 cell-mediated and to a lesser extent, Th2 cell-mediated airway hyperresponsiveness (FIGS. 11A and 11B, respectively). While evidence is emerging that IL-13 mediates Th2-dependent airway hyperresponsiveness (Cohn et al. (1997) supra; Wills-Karps et al. (1998) Science 282:2258-2261), the mechanisms underlying this Th1-mediated lung inflammation is unknown, although previous studies have demonstrated that airway exposure to LPS, which results in neutrophil accumulation in the lungs also results in airway hyperresponsiveness (Lefort et al. (1998) J. Immunol. 161:474-480) in the absence of either IL-4, IL-5, or IL-13 and eosinophilic inflammation. Whatever the precise mechanisms involved, this system has been used to dissect the relative contribution of CD28/ICOS to Th1- and Th2-mediated alterations in airway responsiveness to inhaled methacholine. CTLA-4-Ig treatment suppressed both Th1- and Th2-mediated heightened airway sensitivity consistent with the effects of blockade of CD28 on Th1- and Th2-mediated inflammation. In contrast, ICOS-Ig suppressed only Th2 cell-mediated airway hyperresponsiveness, in the absence of any suppressive effect of Th1-mediated airway hyperresponsiveness (FIG. 11).

These studies provide direct evidence for the existence of Th2 subset specific costimulation mediated via the inducible ICOS gene. Although clonal expansion of natve T cells may be limited in the absence of CD28, effector responses can still develop as suggested by recent observations, demonstrating that despite limited clonal expansion in anergic cells, T helper effector function is not prevented (Malvey et al. (1998) J. Immunol. 5:2168-2177). It is proposed that in the absence of CD28, ICOS engagement on Th2 effector cells would be used as an alternative signaling pathway to CD28. Finally, these data highlight the therapeutic benefit of selectively dampening down inappropriate Th2 responses via suppression of mICOS in diseases, e.g. asthma and allergy, while protective Th1 immunity against bacteria and intracellular parasites remains intact.

Those skilled in the art will recognize, or be able to ascertain using no more than routine experimentation, many equivalents to the specific embodiments of the invention described herein. Such equivalents are intended to be encompassed by the following claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. An isolated nucleic acid molecule comprising the nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, the cDNA insert of the plasmid deposited with ATCC as Accession Number 203302, the cDNA insert of the plasmid deposited with ATCC as Accession Number 203569, the cDNA insert of the plasmid deposited with ATCC as Accession Number 203797, or a complement thereof.

2. The nucleic acid molecule of claim 1 further comprising vector nucleic acid sequences.

3. The nucleic acid molecule of claim 1 further comprising nucleic acid sequences encoding a heterologous polypeptide.

4. A host cell which contains the nucleic acid molecule of claim 1.

5. The host cell of claim 4 which is a mammalian host cell.

6. A nonhuman mammalian host cell containing the nucleic acid molecule of claim 1.

7. An isolated polypeptide comprising the amino acid sequence of SEQ ID NO: 14 or an amino acid sequence encoded by the cDNA insert of the plasmid deposited with ATCC as Accession Number 203569.

8. The polypeptide of claim 7 further comprising heterologous amino acid sequences.

9. A method for producing a polypeptide comprising the amino acid sequence of SEQ ID NO: 14 or an amino acid sequence encoded by the cDNA insert of the plasmid deposited with ATCC as Accession Number 203569, said method comprising culturing a host cell which contains a nucleic acid molecule comprising the nucleotide sequence of SEQ ID NO: 13 or the cDNA insert of the plasmid deposited with ATCC as Accession Number 203569 under conditions in which the nucleic acid molecule is expressed.

10. A method for detecting the presence of a polypeptide of claim 7 in a sample, comprising:
a) contacting the sample with a compound which selectively binds to a polypeptide of claim 7; and
b) determining whether the compound binds to the polypeptide in the sample.

11. The method of claim 10, wherein the compound which binds to the polypeptide is an antibody.

12. A method for identifying a compound which binds to a polypeptide of claim 7 comprising the steps of:
a) contacting a polypeptide, or a cell expressing a polypeptide of claim 7 with a test compound; and
b) determining whether the polypeptide binds to the test compound.

13. The method of claim 12, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for Th2-specific-mediated signal transduction.

14. A method for modulating the activity of a polypeptide of claim 7 comprising contacting the polypeptide or a cell expressing the polypeptide with a compound which binds to the polypeptide in a sufficient concentration to modulate the activity of the polypeptide.

15. A method for identifying a compound which modulates the activity of a polypeptide of claim 7, comprising:
a) contacting a polypeptide of claim 7 with a test compound; and
b) determining the effect of the test compound on the activity of the polypeptide to thereby identify a compound which modulates the activity of the polypeptide.

16. A method for modulating a Th2 response in a mammal, said method comprising administering to said mammal a therapeutically effective amount of a polypeptide or its corresponding antibody, wherein said polypeptide is selected from the group consisting of:
a) a fragment of a polypeptide comprising the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16, wherein the fragment comprises at least 15 contiguous amino acids of SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16;
b) a naturally occurring allelic variant of a polypeptide comprising the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, an amino acid sequence encoded by the cDNA insert of the plasmid deposited with ATCC as Accession Number 203302, an amino acid sequence encoded by the cDNA insert of the plasmid deposited with ATCC as Accession Number 203569, or an amino acid sequence encoded by the cDNA insert of the plasmid deposited with ATCC as Accession Number 203797, wherein the polypeptide is encoded by a nucleic acid molecule which hybridizes to a nucleic acid molecule comprising SEQ ID NO: 1, SEQ ID NO: 3, SEQ ID NO: 5, SEQ ID NO:7, SEQ ID NO: 9, SEQ ID NO: 11, SEQ ID NO: 13, SEQ ID NO: 15, or a complement thereof under stringent conditions; and
c) a polypeptide which is encoded by a nucleic acid molecule comprising a nucleotide sequence which is at least 45% identical to a nucleic acid comprising the nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, or a complement thereof

17. A method for modulating an immune response in a mammal, said method comprising altering activity of a polypeptide in said mammal by administering to said mammal a therapeutically effective amount of said polypeptide or an antibody corresponding to said polypeptide or by regulating in said mammal expression of a nucleotide sequence encoding said polypeptide, wherein said polypeptide is selected from the group consisting of:
a) a fragment of a polypeptide comprising the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16, wherein the fragment comprises at least 15 contiguous amino acids of SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16;
b) a naturally occurring allelic variant of a polypeptide comprising the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, an amino acid sequence encoded by the cDNA insert of the plasmid deposited with ATCC as Accession Number 203302, an amino acid sequence encoded by the cDNA insert of the plasmid deposited with ATCC as Accession Number 203569, or an amino acid sequence encoded by the cDNA insert of the plasmid deposited with ATCC as Accession Number 203797, wherein the polypeptide is encoded by a nucleic acid molecule which hybridizes to a nucleic acid molecule comprising SEQ ID NO: 1, SEQ ID NO: 3, SEQ ID NO: 5, SEQ ID NO:7, SEQ ID NO: 9, SEQ ID NO: 1, SEQ ID NO: 13, SEQ ID NO: 15, or a complement thereof under stringent conditions; and
c) a polypeptide which is encoded by a nucleic acid molecule comprising a nucleotide sequence which is at least 45% identical to a nucleic acid comprising the nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, or a complement thereof

18. A method for suppressing airway inflammation in a mammal, said method comprising altering activity of a polypeptide in said mammal by administering to said mammal a therapeutically effective amount of an antibody corresponding to said polypeptide or by regulating in said mammal expression of a nucleotide sequence encoding said polypeptide, wherein said polypeptide is selected from the group consisting of:
a) a fragment of a polypeptide comprising the amino acid sequence of SEQ ID NO: 2, SEQ ID NO: 4, SEQ ID NO: 6, SEQ ID NO: 8, SEQ ID NO: 10, SEQ ID NO:12, SEQ ID NO: 14, or SEQ ID NO: 16, wherein the fragment comprises at least 15 contiguous amino acids of SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16;
b) a naturally occurring allelic variant of a polypeptide comprising the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, an amino acid sequence encoded by the cDNA insert of the plasmid deposited with ATCC as Accession Number 203302, an amino acid sequence encoded by the cDNA insert of the plasmid deposited with ATCC as Accession Number 203569, or an amino acid sequence encoded by the cDNA insert of the plasmid deposited with ATCC as Accession Number 203797, wherein the polypeptide is encoded by a nucleic acid molecule which hybridizes to a nucleic acid molecule comprising SEQ ID NO: 1, SEQ ID NO: 3, SEQ ID NO: 5, SEQ ID NO:7, SEQ ID NO: 9, SEQ ID NO: 11, SEQ ID NO: 13, SEQ ID NO: 15, or a complement thereof under stringent conditions; and
c) a polypeptide which is encoded by a nucleic acid molecule comprising a nucleotide sequence which is at least 45% identical to a nucleic acid comprising the nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, or a complement thereof.

19. A method for suppressing airway hyperresponsiveness in a mammal, said method comprising altering activity of a polypeptide in said mammal by administering to said mammal a therapeutically effective amount of an antibody corresponding to said polypeptide or by regulating in said mammal expression of a nucleotide sequence encoding said polypeptide, wherein said polypeptide is selected from the group consisting of:
a) a fragment of a polypeptide comprising the amino acid sequence of SEQ ID NO: 2, SEQ ID NO: 4, SEQ ID NO: 6, SEQ ID NO: 8, SEQ ID NO: 10, SEQ ID NO:12, SEQ ID NO: 14, or SEQ ID NO: 16, wherein the fragment comprises at least 15 contiguous amino acids of SEQ ID NO: 2, SEQ ID NO: 4, SEQ ID NO: 6, SEQ ID NO: 8, SEQ ID NO: 10, SEQ ID NO: 12, SEQ ID NO: 14, or SEQ ID NO: 16;
b) a naturally occurring allelic variant of a polypeptide comprising the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, an amino acid sequence encoded by the cDNA insert of the plasmid deposited with ATCC as Accession Number 203302, an amino acid sequence encoded by the cDNA insert of the plasmid deposited with ATCC as Accession Number 203569, or an amino acid sequence encoded by the cDNA insert of the plasmid deposited with ATCC as Accession Number 203797, wherein the polypeptide is encoded by a nucleic acid molecule which hybridizes to a nucleic acid molecule comprising SEQ ID NO: 1, SEQ ID NO: 3, SEQ ID NO: 5, SEQ ID NO:7, SEQ ID NO: 9, SEQ ID NO: 11, SEQ ID NO: 13, SEQ ID NO: 15, or a complement thereof under stringent conditions; and
c) a polypeptide which is encoded by a nucleic acid molecule comprising a nucleotide sequence which is at least 45% identical to a nucleic acid comprising the nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, or a complement thereof.


SEQUENCE LISTING (24 sequences)

SEQ ID NO: 1 - DNA, 3631 bp, Mus sp.
  CDS (126)..(1304)
ccacgcgtccgcggacgcgtgggcgggaaaggaatcctggggttttctagtatccccttctttttttcttttttcttttctttttctttttggccctgcgggcttcagactcagggaacttgctcatggcttttttgatgaagaagaagaaatttaaatttcaaaccactttcaccttggaggagctgactgcggtccccttcgtgaacggggtcctcttttgcaaggtccggctgctggatggcggggattttgtcagcttgtcgtccagggaggaggtgcaggagaactgtgtacgatggcggaagaggttcacctttgtgtgtaaaatgagtgccaacccggccacaggcctgctggacccctgcatctttcgagtgtctgtgcgcaaggagctgaaaggtgggaaggcgtattccaagctgggcttcactgacttgaacctagccgagtttgcaggctcaggctccaccgtccgctgctgcctattggaaggatacgacaccaagaatacccgccaggacaactccatcctcaaggtcaccattggcatgttcctgctctctggggacccctgtttcaagacgccaccgtctaccgccaaatccatctccatcccgggccaggactcctccctgcagctgacgtgtaaaggtggtgggaccagcagcggtggcagcagcagcaccaattccctaactggatcccggccccccaagacccggcccaccatcctgggctcaggactaccagaggagccagaccagagcctgtccagtcctgaggaggtgttccactctggccactcccgcaactccagctatgccagccagcagtccaagctatctggctacagtacagagcactctcgctcctccagcctgtctgacttgacacaccgcagaaatacatccaccagcagcagcgcctctggtggcctcagtatggctgtagagggtcctgagggcatggagagggagcatcggccctctgagaagccacctcggcctcctgagaagcccccacgccccccacgccccttgcatctatcagatcgctcttttcggcgaaagaaagactctgtggagagccacccaacctgggtagatgacactcgaattgatgcagacgacattgtggagaagataatgcagagccaggacttcactgatggcagcaacactgaggacagtaaccttcggctgttcgtgagccgtgatggctccaccacactgagcggcattcagctgggcaacagagtctcctcgggagtctacgagccagtcgtaattgagagccactgagggacccaggagtccggctgaagaagggtcctgccatctctggcacccaggcccgtggcattcctggaggatctgctctgcattgcatctgtgcctcctcttactctagccacactggcccccaaagcgtcattccattttcctcccatccaccctggaaccactggtcctgggtgccactgtgaatattgtcctccgaaccactagaaaatggggcaagatggagctggccaggactgccctgactggagaggacctgggcccagcaaaggacacagccattccatgttgggtgcttgtggcctgccccatccctccatcccccacgacccacttcaggagcagcagcttcagccaggctgacggggcacttgctccattgtccctgcccttcacgagctgggccaccccagaaccctgccacctgtgaccctctgcttactgcttcactgtcttttctctatcctgaggagacctgcaggcagctctctcctgggagctaagacctcaaaccccacactctggattgagaccccacctccttaactgtcctcctgctgccctgcagcctgcactttgcacatgcttgtccccagcacagccaccggcccttgcccttctccagacctcctgggtgctgtctgctgtgcaaccagacctagggtacagtcgcccggctggaacgtggcactaccccttccctctaattaaccccaaactcagacccaagaccggagcctagaagggcctgagcatcatggttccatctgcccagggctgggtcctgagcagctggccttcctgcaggaaaggaaggacgaggcccagccgtgctgggtaagcaaaggccttttccccctgctctcatgtcagccggcctgggctgggttccaggctagagagtcagccaggtggcctgtgactgggtgaactggagctggcttcctgccagacgagcctcagccctcctgggcaaggggtgtgctttccctggggagtcttccccctactactcaccctcttggtacctccccaaccccagcactttctgtcctataatttgcttaaggagagtataggctgccagctcagccctggataaattcccacccccttaagtttcactcacttcgtatcaacccagcaggctggtgtttccttggccccttaaggtgtttttcctcctgagttcacagttcttgtattccttccaccccaacaaggggagataggtgcctctgttccccattccacctgcaccccagcagggctggggataggcgagtatgggacattgtttctgtgggtggggatggctggactccccaggttgggaaggtccttgcctagtttgtgtcttcctcagctagcaaaagttggggctgtggtcttggagctgcctggtgtcaggaccagatcatttctataggttctgtagccgttgttttaatcagcagatgaatgttagaagatgctgccaaggcaaggcagaacatggctagagaaggttccggcagctcagattccatcctgcccagcaggtgcctggcactttaagggactctggcagctctgttcctaggtgaggtgaccaccagaagatggacagagccctagtgtgcttgtctactaccttccccagtcagcattgttttggtctagttctaaattccagggctccaaaccaagcagttggtactgtcaggaagactgccccaaaccaagagcccctgcctaactcaagccaaggtctgggactggatggccactccagtagacgagagctgccacatcctctgcttccaggcactgggccaggataacttagctgaaaacatgaccacaaccaaatgaacactggtggcagccagaggctaagagcctttcctcctgcgctcagtgatccccacgggtgcccagagcacctggacttcaaattctatatacaaagatagaggatatattagaggtatttttgaaaaggaattggtctatgcaatgccagtttgggacctgtaagacagtttaatgtttctattagagagatataaaagatctgcaatatttttagggttcttttctggttaccctacagctgaccacgtggtctggcaggatgagcaggcccatcttcttctgtggggagaatttgcttttgtgttattcctttctaaccaggttaccactgcaccccattccgtgggctgttcatatctgtctcagcttataatagaaaattatgtcatatggggaaatcagcctagtgtcaatgtttggtttgggggtggaattaaatgccgatgtttttgttaaaaaaaaaaaaaaaa

SEQ ID NO: 2 - protein, 392 aa, Mus sp.
MAFLMKKKKFKFQTTFTLEELTAVPFVNGVLFCKVRLLDGGDFVSLSSREEVQENCVRWRKRFTFVCKMSANPATGLLDPCIFRVSVRKELKGGKAYSKLGFTDLNLAEFAGSGSTVRCCLLEGYDTKNTRQDNSILKVTIGMFLLSGDPCFKTPPSTAKSISIPGQDSSLQLTCKGGGTSSGGSSSTNSLTGSRPPKTRPTILGSGLPEEPDQSLSSPEEVFHSGHSRNSSYASQQSKLSGYSTEHSRSSSLSDLTHRRNTSTSSSASGGLSMAVEGPEGMEREHRPSEKPPRPPEKPPRPPRPLHLSDRSFRRKKDSVESHPTWVDDTRIDADDIVEKIMQSQDFTDGSNTEDSNLRLFVSRDGSTTLSGIQLGNRVSSGVYEPVVIESH

SEQ ID NO: 3 - DNA, 1587 bp, Mus sp.
  CDS (36)..(1430)
cggcgcgggtagagggcggtgggcggcgagcggcgatgggccgcgcctggggcttgctcgttggactcctgggcgtcgtgtggctgctgcgcttgggccacggcgaggagcggcggccggagacagcggcacagcgctgcttctgccaggttagtggttacctggacgactgtacctgtgatgtcgagaccatcgataagtttaataactacagacttttcccaagactacaaaagcttcttgaaagtgactactttagatattacaaggtgaacttgaagaagccttgtcctttctggaatgacatcaaccagtgtggaagaagagactgtgccgtcaaaccctgccattctgatgaagttcctgatggaattaagtctgcgagctacaagtattctgaggaagccaaccgcattgaagaatgtgagcaagctgagcgacttggagccgtggatgagtctctgagtgaggagacccagaaagctgtacttcagtggaccaagcatgatgattcgtcagacagcttctgcgaaattgacgatatacagtcccccgatgctgagtatgtggacttactccttaaccctgagcgctacacaggctacaaggggccagacgcttggaggatatggagtgtcatctatgaagaaaactgttttaagccacagacaattcaaaggcctttggcttctgggcgaggaaaaagtaaagagaacacattttacaactggctagaaggcctctgtgtagaaaagagagcattctacagacttatatctggcctgcacgcaagcattaatgtgcatttgagtgcaaggtatcttttacaagatacttggctggaaaagaaatggggtcacaatgtcacagagttccagcagcgctttgatgggattctgactgaaggagaaggcccacgaaggctgaggaacttgtacttcctgtacctgatagagttaagggctctctccaaagtgcttccattttttgagcgtccagattttcagctcttcactgggaataaagttcaggatgcagaaaacaaagcgttacttctggagatacttcatgaaatcaagtcatttcctttgcacttcgatgagaattctttttttgctggggataaaaacgaagcacataaactaaaggaggacttccggctacactttaggaacatttcaagaatcatggactgtgttggctgcttcaagtgccgcctgtggggcaagcttcagacgcaggggctgggcactgctctgaagatcttgttttccgaaaaactgatcgcaaatatgccggaaagcggaccaagttatgagttccagctaaccagacaagaaatagtatcactgtttaatgcatttggaaggatttccacaagtgtgagagaactagagaacttcaggcacttgttacagaatgttcactgaggaggacggttggaatgtgcctgtttctgcacaggggaatctgaagggcagaatctctctgagcaccatggttgcaatgactgtcctaagccaaacgtttatataaagctgcttttgtaaaagaagagatacattgtcttaagtaaatgacattttt

SEQ ID NO: 4 - protein, 464 aa, Mus sp.
MGRAWGLLVGLLGVVWLLRLGHGEERRPETAAQRCFCQVSGYLDDCTCDVETIDKFNNYRLFPRLQKLLESDYFRYYKVNLKKPCPFWNDINQCGRRDCAVKPCHSDEVPDGIKSASYKYSEEANRIEECEQAERLGAVDESLSEETQKAVLQWTKHDDSSDSFCEIDDIQSPDAEYVDLLLNPERYTGYKGPDAWRIWSVIYEENCFKPQTIQRPLASGRGKSKENTFYNWLEGLCVEKRAFYRLISGLHASINVHLSARYLLQDTWLEKKWGHNVTEFQQRFDGILTEGEGPRRLRNLYFLYLIELRALSKVLPFFERPDFQLFTGNKVQDAENKALLLEILHEIKSFPLHFDENSFFAGDKNEAHKLKEDFRLHFRNISRIMDCVGCFKCRLWGKLQTQGLGTALKILFSEKLIANMPESGPSYEFQLTRQEIVSLFNAFGRISTSVRELENFRHLLQNVH

SEQ ID NO: 5 - DNA, 4382 bp, Mus sp.
  CDS (36)..(1430)
cggcgcgggtagagggcggtgggcggcgagcggcgatgggccgcgcctggggcttgctcgttggactcctgggcgtcgtgtggctgctgcgcttgggccacggcgaggagcggcggccggagacagcggcacagcgctgcttctgccaggttagtggttacctggacgactgtacctgtgatgtcgagaccatcgataagtttaataactacagacttttcccaagactacaaaagcttcttgaaagtgactactttagatattacaaggtgaacttgaagaagccttgtcctttctggaatgacatcaaccagtgtggaagaagagactgtgccgtcaaaccctgccattctgatgaagttcctgatggaattaagtctgcgagctacaagtattctgaggaagccaaccgcattgaagaatgtgagcaagctgagcgacttggagccgtggatgagtctctgagtgaggagacccagaaagctgtacttcagtggaccaagcatgatgattcgtcagacagcttctgcgaaattgacgatatacagtcccccgatgctgagtatgtggacttactccttaaccctgagcgctacacaggctacaaggggccagacgcttggaggatatggagtgtcatctatgaagaaaactgttttaagccacagacaattcaaaggcctttggcttctgggcgaggaaaaagtaaagagaacacattttacaactggctagaaggcctctgtgtagaaaagagagcattctacagacttatatctggcctgcacgcaagcattaatgtgcatttgagtgcaaggtatcttttacaagatacttggctggaaaagaaatggggtcacaatgtcacagagttccagcagcgctttgatgggattctgactgaaggagaaggcccacgaaggctgaggaacttgtacttcctgtacctgatagagttaagggctctctccaaagtgcttccattttttgagcgtccagattttcagctcttcactgggaataaagttcaggatgcagaaaacaaagcgttacttctggagatacttcatgaaatcaagtcatttcctttgcacttcgatgagaattctttttttgctggggataaaaacgaagcacataaactaaaggaggacttccggctacactttaggaacatttcaagaatcatggactgtgttggctgcttcaagtgccgcctgtggggcaagcttcagacgcaggggctgggcactgctctgaagatcttgttttccgaaaaactgatcgcaaatatgccggaaagcggaccaagttatgagttccagctaaccagacaagaaatagtatcactgtttaatgcatttggaaggatttccacaagtgtgagagaactagagaacttcaggcacttgttacagaatgttcactgaggaggacggttggaatgtgcctgtttctgcacaggggaatctgaagggcagaatctctctgagcaccatggttgcaatgactgtcctaagccaaacgtttatataaagctgcttttgtaaaagaagagatacattgtcttaagtaaatgacatttttaaaaattgtgttcatgtttaatattattgtgaatagaagtagtattttgataatatacaaattttaatactaagcaaaagtaaggtcattaaactcccttatgataaagctaggtaaatattgccttaggctctcaaactagagttgaatttaagaaggtaatgaaagctaagctaaggtacctgtgacaagtgtttggtaataaaatactaagaggctcagcagtaaaccatggttatcagcctcagataccaatgctttttcatttgcctcatatacaaagaaaatacagactgcccagttagatttaagttttagaaaagcaaccagttttcctttcaatgttaagtgtgttgaagtttattagggcctatttatactacaagttatttgctcgaagttcaagtttaactgggagccctgtgttttcctggcaaccttaaaatacactggtatgaaacattccttttagagttaaatagctattttgattgtgctgttttggtatgttgggaagtagaacaattcaaaggcacaggagatttctaaacatcgtagaaagataaatagattatatatttattctcataatagtttccctaatggaattttggggaaaattttatttttatataatttcatatttacagaaaagtttcaaaaacagttaaaaaaaaaaccaaactggttttactcagattcatggattgttcacatgtgttttaacccctcaccccagctcagagtggcgattgagcccaggacctcagtcatgctgggctagccctgcattcatgagctgtgtcctcaggccttgcttaagctcctacatagacatagacaccctgtttttatttaagggtttgagtcatgggcctcatgatttaacccaaaatacttcagtgtggtggtcaataatttcctaaaaactattttttgttttctttttaaaggaatatccaaaccttacaaatctgagctgaactacttactataagccttaaaataagggagacatcccacatcccagccacctgacgtttgaatttggttgcctttttcgacctgtgtagtcacaggctatacaactcatgtttttacgcatgaaactctgtttttctccacaggatggagctgtaacgtagataggatgggttgttagtagaagctctacactgagagcagtatttgatgtggcttaagaatttacatcacatggcaattataaatggcatatacctcaaaattatgcccccttgaggtcacatctaatgtcttgagcagttaggactggttgagatgtcacataagaaaaagcaggacttacgtatgtaatggctaggaatgtcactttacactgttgtgtattttctctgtccctaagacttggtctagtgccaggcatacagttggtgtctaatatttattgatggaaagtatatgtatttagtatacgttaagtgatggtgatgtagcttgtttagtaatgtatcctatttccccctccctctcactttttcttttggatctcataataaaagacatcaacccactaattaggtcagttttcttggggacttaaaagaactaaagtgatcaccacacacaagccagcccttttgaaaccctcactgctttcacgtgcttttcccacttgattggctatgtcctctgtccctccaagtgaagtctaatacctaattctaagtttatagttaacaagcagtttaatttttaagaaatcttaacttttcctgtacatgacacaattgacaatccctattcctttgtgtccttgtgtttggattactcctgggtatctttcttctggtctcttcatttaacctaagtatcattcctcagactttttgctcttaagtcactggaccgtacttttgagtaatttcaatgcagtaatctcattttagcttctcagtctgtctctctgctgtgctcacttggctgcctgttgtcgcttctgcatcttcaaggactcagtcaccacaaacctactctatgtttgtgttccttacttggttaatttcagagccgttgcaccgttcaaggctttacagttcattcctgtatcgtccacctgactattggtgtcttaagctctgccgattttctctcaacatttctatcagtgtttttcagtttttattacgatcaccaaaggattacctgtcttttcccatctctctgttggctcctatcaccagattgggctttctagaaaccttcagcctaattcctgctggcaagtttcatgagctttttggtgagagtctctgcttgatatccaagaccctcctcttgtgtttgccactgttctgataagagtgttaaaacgttttccaaaagtccagagttgttggccttccaaacctttgcaaacacagtatttgcattgagagccctaggcttctttctcatctgggaaattccatgcatttttgaaatctgtgaaattacccctgtccccttcctatggtcaccactgtaactgcaacctacctctactgaatcacttattgtactgtatattttattttatgaagtgtcctttattagaatgtgagctccttaggggcaggaaaagaaactattcattttggcacctctatagcataatgaatagtttcttttcctgcccctaaggagctcacattctaataaaggacacttcataaaataaaatatacagtacaataagtgattcagtagaggtaggttgcagttacagtggtgaccataggaaggggacaggggtaatttcacagatttcaaaaatg

SEQ ID NO: 6 - protein, 464 aa, Mus sp.; identical to SEQ ID NO: 4

SEQ ID NO: 7 - DNA, 2080 bp, Mus sp.
  CDS (46)..(648)
gtcgacccacgcgtccggagaagactgcaactgctcctggcagacatgaagccgtacttctgccatgtctttgtcttctgcttcctaatcagacttttaacaggagaaatcaatggctcggccgatcataggatgttttcatttcacaatggaggtgtacagatttcttgtaaataccctgagactgtccagcagttaaaaatgcgattgttcagagagagagaagtcctctgcgaactcaccaagaccaagggaagcggaaatgcggtgtccatcaagaatccaatgctctgtctatatcatctgtcaaacaacagcgtctcttttttcctaaacaacccagacagctcccagggaagctattacttctgcagcctgtccatttttgacccacctccttttcaagaaaggaaccttagtggaggatatttgcatatttatgaatcccagctctgctgccagctgaagctctggctacccgtagggtgtgcagctttcgttgtggtactcctttttggatgcatacttatcatctggttttcaaaaaagaaatacggatccagtgtgcatgaccctaatagtgaatacatgttcatggcggcagtcaacacaaacaaaaagtctagacttgcaggtgtgacctcataagctggaacacccggacccacagaggaaacgcatggactagttcccctgaaacttgaatggagaaattcttctattttctggaccacagggcacctgacttgatttaactacagacaacattctgctggtgttttgtttgtctggatcagtgactatcagtcattcagaatttcagcagactgccctgggtttgctgagtccttttaagatgaaccccttcttaaggaagacccagttcatatatattcaacaaacagacctcactgggatagaatgcccctctctccgtgcctgattctagctatgcactggccaggaaaacaaacatatctccagcatttttttacaaaaaatgccaagggtatgaatctgtaaagcacacaggcagccaatggccaccatctgtcctcatttttcagattccattttctttccatggagatcagcattctttctagaatcagacagtagagggagatgcttcacaacagaagcgcttgtgtttctgcgattttgatgtatatactttatcgaactcatgcattagaaccaacatgctctcttccaattcctagactctagctgatcactgcttcagggcttagatgcctgctcttgccttcaaatttccccttaaagatatgtccacatgtctacttggcagcctgctgccactccaaataggaagctcagcctacattccctcccacccctgttgctcaaaaaaaaaaattagtagatatgatgttcccatattctccctgccaaagtaattttttccagaaaagacatctaaattcagttaatatggtttactatgttgatattagtggtagtaaacatttctcagaaacaaaagcaaattaattttgctgtggtgttttctactattatctgtgtttccatggtgctattaatcacaagcttagctatttttgtagatcatattaaagttgcaagcaaggaaagcaaccctctgttaatgggcaaacattctcctggggtagaatgaattatctatttagcctgaaagctgcggtttctgggtggtggctgccagactacagccacactttgctctctctgagtttgacaggttgaagtagtccccctggagcagcactccaggctacgctggagtcccagagttgggagatgcctgggacaagctgctgctttggtctctgggatctgggaagtacagtagggccaagacagaattccctctcctagaaactatgcagcctggaagtcagccctagcactttaagatagccttctttagaatatgagttagttggaaggattctgaagtgtaaagagcatatgactgctctgagctggaccattttctctactttcctgtctgtatgcctaagacttctggagcagccaatgtgtatgcaacataaaaaaaaaaaaaaaaaaaaaa

SEQ ID NO: 8 - protein, 200 aa, Mus sp.
MKPYFCHVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 9 - DNA, 3266 bp, Mus sp.
  CDS (40)..(642)
  unsure (1)..(3266): n= a,  g,  c,  or t
ccacgcgtccggagaagactgcaactgctcctggcagacatgaagccgtacttctgccatgtctttgtcttctgcttcctaatcagacttttaacaggagaaatcaatggctcggccgatcataggatgttttcatttcacaatggaggtgtacagatttcttgtaaataccctgagactgtccagcagttaaaaatgcgattgttcagagagagagaagtcctctgcgaactcaccaagaccaagggaagcggaaatgcggtgtccatcaagaatccaatgctctgtctatatcatctgtcaaacaacagcgtctcttttttcctaaacaacccagacagctcccagggaagctattacttctgcagcctgtccatttttgacccacctccttttcaagaaaggaaccttagtggaggatatttgcatatttatgaatcccagctctgctgccagctgaagctctggctacccgtagggtgtgcagctttcgttgtggtactcctttttggatgcatacttatcatctggttttcaaaaaagaaatacggatccagtgtgcatgaccctaatagtgaatacatgttcatggcggcagtcaacacaaacaaaaagtctagacttgcaggtgtgacctcataagctggaacacccggacccacagaggaaacgcatggactagttcccctgaaacttgaatggagaaattcttctattttctggaccacagggcacctgacttgatttaactacagacaacattctgctggtgttttgtttgtctggatcagtgactatcagtcattcagaatttcagcagactgccctgggtttgctgagtccttttaagatgaaccccttcttaaggaagacccagctcatatatattcaacaaacagacctcactgggatagaatgcccctctctccgtgcctgattctagctatgcactggccaggaaaacaaacatatctccagcatttttttacaaaaaatgccaagggtatgaatctgtaaagcacacaggcagccaatggccaccatctgtcctcatttttcagattccattttctttccatggagatcagcattctttctagaatcagacagtagagggagatgcttcacaacagaagcgcttgtgtttctgcgattttgatgtatatactttatcgaactcatgcattagaaccaacatgctctcttccaattcctagactctagctgatcactgcttcagggcttagatgcctgctcttgccttcaaaatttccccttaaagatatgtccacatgtctacttggcagcctgctgccactccaaataggaagctcagcctacattccctcccacccctgttgctcaaaaaaaaaaattagtagatatgatgttcccatattctccctgccaaagtaattttttccagaaaagacatctaaattcagttaatatggtttactatgttgatattagtggtagtaaacatttctcagaaacaaaagcaaattaattttgctgtggtgttttctactattatctgtgtttccatggtgctattaatcacaagcttagctatttttgtagatcatattaaagttgcaagcaaggaaagcaaccctctgttaatgggcaaacattctcctggggtagaatgaattatctatttagcctgaaagctgcggtttctgggtggtggctgccagactacagccacactttgctctctctgagtttgacaggttgaagtagtccccctggagcagcactccaggctacgctggagtcccagagttgggagatgcctgggacaagctgctgctttggtctctgggatctgggaagtacagtagggccaagacagaattccctctcctagaaactatgcagcctggaagtcagccctagcactttaagatagccttctttagaatatgagttagttggaaggattctgaagtgtaaagagcatatgactgctctgagctggaccattttctctactttcctgtctgtatgcctaagacttctggagcagccaatgtgtatgcaacataaaaaaacaaaaaacaaaaaaactcacaaactttaagtaaaatatggctgcatcatagttttaagttatagttgcttacttacagtttactttaaaagtgagctgtgcatcagtatatagttattacgttaagcatgtgtaatgctggctatgtacagtacagtactgaacgtgtaatttgaatcaagtatggtgttctctctcttcagatgacccgggcagtctgactggctttgttagggcattccccaagttggacggtcctttctgtgtgagggtgaaggtgctttactctgttccttgaaggatccccattcccaacacgaacacctaacaatgacttcttgaagaacaaaatttctgtgaaataaaatctggcttaaagaggagccacttgctaaaagatgttagcaataatacaaagtaaaggctaagcacttgtgtgcagcaatgggctataaccttgctgtgttttctgggggtgaagaagtgatgaccttagctaccatatgcaaattagcccttgctctccaatgacctttcgtgctacataagatagttgctccctttgtctcgtttttccccttttagactctgaaatattcacaagcttataaaagagtcagaagcacaggaaacagcacgctctgtgcctgagttagtttctgcgttgacctcctggccccacagaatggtgcctatttcctatggaaatgaagactgcactcttgataacttgacagtcaccaatatcagaaaattaacactgatatgtcactaccctctagtctttggtctgcattcaagatcttcccctggcttctaggatgtatgtaggagctgaaagttctgctggggatgtccctttgtgttcgattgacctgtctcgggagtgttcttcagtctagacagttctcttcagcctgtagcctgggcccttttgcagatcacacaccattattttgtagaatgttccttttgtggggggtcacgctgatgtttcttcatgaatgaaatgaggttactcgtctttagcagaaataacacagaagtggtgttgtgccctatagggcggctcacattttggtttgccaagtaactgccagtgtttgttttcatctcctgtcaagcttctccattgtaaagggattctgctcctgaacagccaataaatatgctttgtgagagaaaaaaaaaaaananaaaaaaaaaa

SEQ ID NO: 10 - protein, 200 aa, Mus sp.; identical to SEQ ID NO: 8

SEQ ID NO: 11 - DNA, 2703 bp, Homo sapiens
  CDS (115)..(711)
tccggaattcccgggtcgacccacgcgtccgcggacgcgtgggcggacgcgtgggcggacgcgtgggcggacgcgtgggctgaacgcgaggactgttaactgtttctggcaaacatgaagtcaggcctctggtatttctttctcttctgcttgcgcattaaagttttaacaggagaaatcaatggttctgccaattatgagatgtttatatttcacaacggaggtgtacaaattttatgcaaatatcctgacattgtccagcaatttaaaatgcagttgctgaaaggggggcaaatactctgcgatctcactaagacaaaaggaagtggaaacacagtgtccattaagagtctgaaattctgccattctcagttatccaacaacagtgtctctttttttctatacaacttggaccattctcatgccaactattacttctgcaacctatcaatttttgatcctcctccttttaaagtaactcttacaggaggatatttgcatatttatgaatcacaactttgttgccagctgaagttctggttacccataggatgtgcagcctttgttgtagtctgcattttgggatgcatacttatttgttggcttacaaaaaagtattcatccagtgtgcacgaccctaacggtgaatacatgttcatgagagcagtgaacacagccaaaaaatctagactcacagatgtgaccctataatatggaactctggcacccaggcatgaagcacgttggccagttttcctcaacttgaagtgcaagattctcttatttccgggaccacggagagtctgacttaactacatacatcttctgctggtgttttgttcaatctggaagaatgactgtatcagtcaatggggattttaacagactgccttggtactgccgagtcctctcaaaacaaacaccctcttgcaaccagctttggagaaagcccagctcctgtgtgctcactgggagtggaatccctgtctccacatctgctcctagcagtgcatcagccagtaaaacaaacacatttacaagaaaaatgttttaaagatgccaggggtactgaatctgcaaagcaaatgagcagccaaggaccagcatctgtccgcatttcactatcatactacctcttctttctgtagggatgagaattcctcttttaatcagtgaagggagatgcttcaaagctggagctattttatttctgagatgttgatgtgaactgtacattagtacatactcagtactctccttcaattgctgaaccccagttgaccattttaccaagactttagatgctttcttgtgccctcaattttctttttaaaaatacttctacatgactgcttgacagcccaacagccactctcaatagagagctatgtcttacattctttcctctgctgctcaatagttttatatatctatgcatacatatatacacacatatgtatataaaattcataatgaatatatttgcctatattctccctacaagaatatttttgctccagaaagacatgttcttttctcaaattcagttaaaatggtttactttgttcaagttagtggtaggaaacattgcccggaattgaaagcaaatttattttattatcctattttctaccattatctatgttttcatggtgctattaattacaagtttagttctttttgtacgatcatattaaaattgcaaacaaaatcatctttaatgggccagcattctcatggggtagagcagaatattcatttagcctgaaagctgcagttactataggttgctgtcagactatacccatggtgcctctgggcttgacaggtcaaaatggtccccatcagcctggagcagccctccagacctgggtggaattccagggttgagagactcccctgagccagaggccactaggtattcttgctcccagaggctgaagtcaccctgggaatcacagtggtctacctgcattcataattccaggatctgtgaagagcacatatgtgtcagggcacaattccctctcataaaaaccacacagcctggaaattggccctggcccttcaagatagccttctttagaatatgatttggctagaaagattcttaaatatgtggaatatgattattcttagctggaatattttctctacttcctgtctgcatgcccaaggcttctgaagcagccaatgtcgatgcaacaacatttgtaactttaggtaaactgggattatgttgtagtttaacattttgtaactgtgtgcttatagtttacaagtgagacccgatatgtcattatgcatacttatattatcttaagcatgtgtaatgctggatgtgtacagtacagtactgaacttgtaatttgaatctagtatggtgttctgttttcagctgacttggacaacctgactggctttgcacaggtgttccctgagttgtttgcaggtttctgtgtgtggggtggggtatggggaggagaaccttcatggtggcccacctggcctggttgtccaagctgtgcctcgacacatcctcatccccagcatgggacacctcaagatgaataataattcacaaaatttctgtgaaatcaaatccagttttaagaggagccacttatcaaagagattttaacagtagtaagaaggcaaagaataaacatttgatattcagcaactgaaaaaaaaaaaaaaaaa

SEQ ID NO: 12 - protein, 198 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKYSSSVHDPNGEYMFMRAVNTAKKSRLTDVTL

SEQ ID NO: 13 - DNA, 1795 bp, Homo sapiens
  CDS (57)..(1502)
gtcgacccacgcgtccgggcctcccaagcgtggggcgacaagctgccggagctgcaatgggccgcggctggggattcttgtttggcctcctgggcgccgtgtggctgctcagctcgggccacggagaggagcagcccccggagacagcggcacagaggtgcttctgccaggttagtggttacttggatgattgtacctgtgatgttgaaaccattgatagatttaataactacaggcttttcccaagactacaaaaacttcttgaaagtgactactttaggtattacaaggtaaacctgaagaggccgtgtcctttctggaatgacatcagccagtgtggaagaagggactgtgctgtcaaaccatgtcaatctgatgaagttcctgatggaattaaatctgcgagctacaagtattctgaagaagccaataatctcattgaagaatgtgaacaagctgaacgacttggagcagtggatgaatctctgagtgaggaaacacagaaggctgttcttcagtggaccaagcatgatgattcttcagataacttctgtgaagctgatgattggatctgctggtggtcatctaagaagaaagtatttgacattcagtcccctgaagctgaatatgtagatttgcttcttaatcctgagcgctacactggttacaagggaccagatgcttggaaaatatggaatgtcatctacgaagaaaactgttttaagccacagacaattaaaagacctttaaatcctttggcttctggtcaagggacaagtgaagagaacactttttacagttggctagaaggtctctgtgtagaaaaaagagcattctacagacttatatctggcctacatgcaagcattaatgtgcatttgagtgcaagatatcttttacaagagacctggttagaaaagaaatggggacacaacattacagaatttcaacagcgatttgatggaattttgactgaaggagaaggtccaagaaggcttaagaacttgtattttctctacttaatagaactaagggccttatccaaagtgttaccattcttcgagcgcccagattttcaactctttactggaaataaaattcaggatgaggaaaacaaaatgttacttctggaaatacttcatgaaatcaagtcatttcctttgcattttgatgagaattcattttttgctggggataaaaaagaagcacacaaactaaaggaggactttcgactgcattttagaaatatttcaagaattatggattgtgttggttgttttaaatgtcgtctgtggggaaagcttcagactcagggtttgggcactgctctgaagatcttattttctgagaaattgatagcaaatatgccagaaagtggacctagttatgaattccatctaaccagacaagaaatagtatcattattcaacgcatttggaagaatttctacaagtgtgaaagaattagaaaacttcaggaacttgttacagaatattcattaaagaaaacaagctgatatgtgcctgtttctggacaatggaggcgaaagagtggaatttcattcaaaggcataatagcaatgacagtcttaagccaaacattttatataaagttgcttttgtaaaggagaattatattgttttaagtaaacacatttttaaaaattgtgttaagtctatgtataatactactgtgagtaaaagtaatactttaataatgtggtacaaattttaaagtttaatattgaataaaaggaggattatcaaattcaaaaaaaaaaaaaaagggcggccgc

SEQ ID NO: 14 - protein, 481 aa, Homo sapiens
MGRGWGFLFGLLGAVWLLSSGHGEEQPPETAAQRCFCQVSGYLDDCTCDVETIDRFNNYRLFPRLQKLLESDYFRYYKVNLKRPCPFWNDISQCGRRDCAVKPCQSDEVPDGIKSASYKYSEEANNLIEECEQAERLGAVDESLSEETQKAVLQWTKHDDSSDNFCEADDWICWWSSKKKVFDIQSPEAEYVDLLLNPERYTGYKGPDAWKIWNVIYEENCFKPQTIKRPLNPLASGQGTSEENTFYSWLEGLCVEKRAFYRLISGLHASINVHLSARYLLQETWLEKKWGHNITEFQQRFDGILTEGEGPRRLKNLYFLYLIELRALSKVLPFFERPDFQLFTGNKIQDEENKMLLLEILHEIKSFPLHFDENSFFAGDKKEAHKLKEDFRLHFRNISRIMDCVGCFKCRLWGKLQTQGLGTALKILFSEKLIANMPESGPSYEFHLTRQEIVSLFNAFGRISTSVKELENFRNLLQNIH

SEQ ID NO: 15 - DNA, 3868 bp, Homo sapiens
  CDS (325)..(1479)
gcggccgctcgcgcgcagccccgcacctccgcccctgcctctgcctcctgggccatgccctgctgtttacatgccggtgaggtccccggccgctccgaacccctccgagccccggctccccgagggtgaagcccgccggcccgcgaactggactggtggatctctcagacctggggccccggactccgatctccgccgtctccgccaccatcagggcgggatccggctctggtgttttgaggagggggtgtggtgtagggaaaggaatcccgtccctctccaccttttttcgccttcggggcttcagactcagggaactcgctcatggctttcttgatgaagaagaagaaattcaaattccaaactactttcaccctggaggagctgactgcggttcccttcgtgaacggggtcctcttctgcaaggtccggctgctggatggaggggattttgtcagcttgtcgtcaagggaggaggtacaggagaactgtgtgcggtggcgaaagaggttcaccttcgtgtgtaagatgagtgctaacccggccaccggcctgctggacccctgtgtcttccgtgtgtctgtgcgcaaggagctgaaaggcgggaaggcttattccaagctgggcttcgctgacttgaacctggccgagtttgcgggctcgggctccacggtgcgctgctgcctgctcgagggatatgacacgaagaacactcgccaggacaactccatccttaaggtcaccattggtatgttcctgctctctggagatccctgcttcaagacgccaccatcgactgccaagtccatctccatcccaggccaggattcctccctgcagctgacgtgtaagggtggtgggaccagcagtgggggcagcagcaccaactccctgactgggtcccggccccccaaggctcggcccactattctcagctcagggctgccagaggaacccgaccagaacctgtccagccctgaggaggtgttccactctggccactcccgcaactccagctatgccagccagcagtccaagatctccggctacagcacagagcactcgcgctcctccagcctctcagacctgacgcaccgccgcaacacgtccaccagcagcagcgcctctgggggccttggcatgaccgtggagggccctgagggcagtgagcgggagcaccggcccccggagaagccgccgcggccaccccggcccctgcatctgtccgatcgctctttcaggcggaagaaggactcggtggagagccacccgacctgggtggacgacacgcggatcgatgcggatgccatcgtggagaagatcgtgcagagccaggacttcacagatggcagcaacaccgaggacagcaacctccggctgttcgtgagccgcgatggctctgccacgctgagcggcatccagcttgccaccagggtctcttctggggtctacgagccagttgtgattgaaagccattgaggagcaggtgtccgggctggagaagagtcctgctttctctggagtccagacctgtatcattccatgaggaactttccccttcagatcacctctgcgccacatctcatccatgcctcctccatgcactccagtccacactccccgtagcatcattccattgcccctcccatccatgctgggaccctcctggcccaccaaggcccaggcaccactgtgaatattctcctctgaaccactagagggcaggccaggcaggccaggcgggcccgtgcagcttgtgggcaagaaggagctggcaaggaccggcgctgctggagactgacccagccctctggctgaggacatgcagcagctcctaaatgtagagatgcctgtggctgagggggcctctctacctgtgtccccactcactccaggagcactggctttggtcacgtcttagcagcagggccttgctccgttgttcccttgccctggtggtgggggggccagaccgcctccggaatcctgccacctgtgactgtctgactgcttagtgcttcagctgtcccttccttgtgtcctgggggacctgctggcggcctcttcctgggagccatgacctcagaccccacccacactccagatcgagacccctgcctccccccggcaaatgtcctcccgctgccttgcagcctgcactttgcacatgctcacccccagcacagtcccactggcccctcacctccccttccctgagctccttcccaaggactcctggtcactgcctgctgtgcagtcagaggcccagggtccagcagcccggcgggaacgggtgctgcctcttcctccagttagctccagctcaggtctgagacccgtgctgagaaaggtctgagcaccgaccgtgccctctgcccagggctgggtcctgagcagctggttttcctgcaggaaggttggagcaagcaaagtccttctctgccctcagggtcagctgcccagactggggcggatgccagagaggcaggtgggctgtggctggactggtccggagctggcttccttaccagaaaagcctcagccttcctctggaagcatcccccgttctgggcaagggggaagggctcctttaaggggtgtgctttcccagtggggagcagtctggccctgccccctactaaagcctctgctctcagcactttcccccaagtccttgtaacttgcttgaaggtgggttctggctgccagccagtccctggacaaactctcctgccccttttaaatttcactcattttgtataaacccagcaggctggtgtttacttagccctgtagcttttttcattttttctttccgtctttcttcttgagttcacggttcaatattgcctcctcgccctggtgaggggaggtgctgcttttctgccccacctgccggctggttccagcagcgctggggcccagctggggggccgggatgggggcttctctctctgggaggggtgcaggtgccctccccaggctgggagggttccttccctagctccccatctgcccccgctggtgagagttgggcttcttggtcttggaactccctggcattgggaacagagcatttccagcatttgttgttgttgttttactcacctaacccttagaaaatgaatgttagaaggtgcctgccgaggcgggacagagtgtttgctcgcgctggagaaggctctgctcagccctgagagtcccttcctgccccaccgatactggcactttaaaaaggaagctgaccgcacagtgtccagacgaattggcccccagaagatggggagttctgtcctgcccttctgtgtctgcgtgacctcacccagcctaggagggaggtgcattcagggtagatttgcctctcattcaaagttctggggctttgggcggaaaacagccagctttggcgctgttggggagactcctccagaccaggaaccccagaaggagacagagcctgccacatcctcccacgccaggccctgggccagggtgattggactgagaatttggccacaaccaaattgatgctggctggaaccagaggccagaaagcctggccttgtccccatgtgggagccctgtcctcagccctcttgtccccttgagctcagtgaattcccaccaggtgcccacagctcctggacttcaaattctatatattgagagagttggagagtatatcagagatatttttggaaaggagttggtctatgcaatgtcagtttggaatcttcttgaaagtttaatgtttttattaggagatttaaagaaaataaaggtctacaatatcaaaaaaaaaacccacgcgtccgcccacgcgtccgcccacgcgtccgcccacgcgtccgcggacgcgtgggtcgac

SEQ ID NO: 16 - protein, 384 aa, Homo sapiens
MAFLMKKKKFKFQTTFTLEELTAVPFVNGVLFCKVRLLDGGDFVSLSSREEVQENCVRWRKRFTFVCKMSANPATGLLDPCVFRVSVRKELKGGKAYSKLGFADLNLAEFAGSGSTVRCCLLEGYDTKNTRQDNSILKVTIGMFLLSGDPCFKTPPSTAKSISIPGQDSSLQLTCKGGGTSSGGSSTNSLTGSRPPKARPTILSSGLPEEPDQNLSSPEEVFHSGHSRNSSYASQQSKISGYSTEHSRSSSLSDLTHRRNTSTSSSASGGLGMTVEGPEGSEREHRPPEKPPRPPRPLHLSDRSFRRKKDSVESHPTWVDDTRIDADAIVEKIVQSQDFTDGSNTEDSNLRLFVSRDGSATLSGIQLATRVSSGVYEPVVIESH

SEQ ID NO: 17 - protein, 442 aa, Trypanosoma brucei
MLKMRLLIVVPVLLGLVWQILLRAELDGVSFFGMYISANNSGAGSYVRTKKGNALKEGFCSLTMDEVSQNTEGITGLLNNITSHPYFRYFKVNLDRECRYWVAEASCTCDSNGCQICTCDDSGIPETLKYPYDMSDVSAVERRTAPDKHAAKGFEDEIKPIDPDRDATYVDLLQNPEANTGYSGPKAARVWQAVYDNCNIDGLPSNDTAGVENREKALLRQLLSGLHTSITMHVAAFFYNDTKGDSPLRSLGVLNNPNISFYPNCGMFRRIVKNDEFIRNLFVVYQFVLRAVAKTKRAFLANSSLYNSGFNGAATDGDVRLYSNIGELFSSKLFRVATFDEQKFFESPGAHLLVRQMKRVVHNVTTLMDCVTCEKCRAWGKLETAALATALKIVFGSAENVVELNRGERVALINFARQLAISVKNVRSLAAVCEKFNYTAMS

SEQ ID NO: 18 - protein, 218 aa, Mus sp.
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQVTTMNMTPRRPGLTRKPYQPYAPARDFAAYRP

SEQ ID NO: 19 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 20 - protein, 223 aa, Mus sp.
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVSAVSLSKMLKKRSPLTTGVYVKMPPTEPECEKQFQPYFIPIN

SEQ ID NO: 21 - protein, 223 aa, Homo sapiens
MACLGFQRHKAQLNLAARTWPCTLLFFLLFIPVFCKAMHVAQPAVVLASSRGIASFVCEYASPGKATEVRVTVLRQADSQVTEVCAATYMTGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 22 - protein, 284 aa, Caenorhabditis elegans
MNFISAKLVFVPWLWNKMAFIKRKTVKFSVDLQVCQLSDVPLVNATVFGKMRLLDGGSFEEATERVEEQKGGKSYYKLGFVDINLSEYAASGVEGISRTYLLNGYTSNQRLDNSKVCIKVAMTHQSADPFFRVPRLSTFGPRQDGAIDQDGFRADDETDSEEGTSSHPKLSNVDVLESSSAASNSQVDEPVVERRVIHPPQHPTCQLRRFSQDRSAQKIQHSRFDADNVIDKIIAECRISEDEVESNSGGLVLEKFLDKHGKPLVNPRQPVKRTSYVAEHFNDA

SEQ ID NO: 23 - DNA, 23 bp, Artificial Sequence
  Description of Artificial Sequence oligonucleotide primer
aaccttctagtcctttggtctgc

SEQ ID NO: 24 - DNA, 18 bp, Artificial Sequence
  Description of Artificial Sequence oligonucleotide primer
ggcccaggctacaggctg